SEQUENCE LISTING (16 sequences)

SEQ ID NO: 1 - protein, 874 aa
MGWPGLRPLLLAGLASLLLPGSAAAGLKLMGAPVKMTVSQGQPVKLNCSVEGMEDPDIHWMKDGTVVQNASQVSISISEHSWIGLLSLKSVERSDAGLYWCQVKDGEETKISQSVWLTVEGVPFFTVEPKDLAVPPNAPFQLSCEAVGPPEPVTIYWWRGLTKVGGPAPSPSVLNVTGVTQRTEFSCEARNIKGLATSRPAIVRLQAPPAAPFNTTVTTISSYNASVAWVPGADGLALLHSCTVQVAHAPGEWEALAVVVPVPPFTCLLRNLAPATNYSLRVRCANALGPSPYGDWVPFQTKGLAPARAPQNFHAIRTDSGLILEWEEVIPEDPGEGPLGPYKLSWVQENGTQDELMVEGTRANLTDWDPQKDLILRVCASNAIGDGPWSQPLVVSSHDHAGRQGPPHSRTSWVPVVLGVLTALITAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLVRFMVDIACGMEYLSSRNFIHRDLAARNCMLAEDMTVCVADFGLSRKIYSGDYYRQGCASKLPVKWLALESLADNLYTVHSDVWAFGVTMWEIMTRGQTPYAGIENAEIYNYLIGGNRLKQPPECMEEVYDLMYQCWSADPKQRPSFTCLRMELENILGHLSVLSTSQDPLYINIERAEQPTESGSPELHCGERSSSEAGDGSGVGAVGGIPSDSRYIFSPGGLSESPGQLEQQPESPLNENQRLLLLQQGLLPHSSC

SEQ ID NO: 2 - protein, 850 aa
AGLKLMGAPVKMTVSQGQPVKLNCSVEGMEDPDIHWMKDGTVVQNASQVSISISEHSWIGLLSLKSVERSDAGLYWCQVKDGEETKISQSVWLTVEGVPFFTVEPKDLAVPPNAPFQLSCEAVGPPEPVTIYWWRGLTKVGGPAPSPSVLNVTGVTQRTEFSCEARNIKGLATSRPAIVRLQAPPAAPFNTTVTTISSYNASVAWVPGADGLALLHSCTVQVAHAPGEWEALAVVVPVPPFTCLLRNLAPATNYSLRVRCANALGPSPYGDWVPFQTKGLAPARAPQNFHAIRTDSGLILEWEEVIPEDPGEGPLGPYKLSWVQENGTQDELMVEGTRANLTDWDPQKDLILRVCASNAIGDGPWSQPLVVSSHDHAGRQGPPHSRTSWVPVVLGVLTALITAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLVRFMVDIACGMEYLSSRNFIHRDLAARNCMLAEDMTVCVADFGLSRKIYSGDYYRQGCASKLPVKWLALESLADNLYTVHSDVWAFGVTMWEIMTRGQTPYAGIENAEIYNYLIGGNRLKQPPECMEEVYDLMYQCWSADPKQRPSFTCLRMELENILGHLSVLSTSQDPLYINIERAEQPTESGSPELHCGERSSSEAGDGSGVGAVGGIPSDSRYIFSPGGLSESPGQLEQQPESPLNENQRLLLLQQGLLPHSSC

SEQ ID NO: 3 - protein, 876 aa
MGRPGLPPLPLPPPPRLGLLLAESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTVGGSLELPGRDQPYSGAGDGSGMGAVGGTPSDCRYILTPGGLAEQPGQAEHQPESPLNETQRLLLLQQGLLPHSSC

SEQ ID NO: 4 - protein, 850 aa
AGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTVGGSLELPGRDQPYSGAGDGSGMGAVGGTPSDCRYILTPGGLAEQPGQAEHQPESPLNETQRLLLLQQGLLPHSSC

SEQ ID NO: 5 - protein, 386 aa
AGLKLMGAPVKMTVSQGQPVKLNCSVEGMEDPDIHWMKDGTVVQNASQVSISISEHSWIGLLSLKSVERSDAGLYWCQVKDGEETKISQSVWLTVEGVPFFTVEPKDLAVPPNAPFQLSCEAVGPPEPVTIYWWRGLTKVGGPAPSPSVLNVTGVTQRTEFSCEARNIKGLATSRPAIVRLQAPPAAPFNTTVTTISSYNASVAWVPGADGLALLHSCTVQVAHAPGEWEALAVVVPVPPFTCLLRNLAPATNYSLRVRCANALGPSPYGDWVPFQTKGLAPARAPQNFHAIRTDSGLILEWEEVIPEDPGEGPLGPYKLSWVQENGTQDELMVEGTRANLTDWDPQKDLILRVCASNAIGDGPWSQPLVVSSHDHAGRQGPPHSR

SEQ ID NO: 6 - protein, 386 aa
AGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSR

SEQ ID NO: 7 - DNA (cDNA), 3919 bp; SINGLE-stranded, LINEAR
ggcacgagtgtggaaggagcgcggtggcccagccgcagccccggggactcctcgctgctgacggcggtggccgcggctctaggcggccgcgggtcccggacgccccggccgagcgccgccccccgcccctcccgcgggcctcccgcccctcctccgccaccctcctctcagcgctcgcgggccgggcccggcatggtgcggcgtcgccgccgatggcgctgaggcggagcatggggtggccggggctccggccgctgctgctggcgggactggcttctctgctgctccccgggtctgcggccgcaggcctgaagctcatgggcgccccagtgaagatgaccgtgtctcaggggcagccagtgaagctcaactgcagcgtggaggggatggaggaccctgacatccactggatgaaggatggcaccgtggtccagaatgcaagccaggtgtccatctccatcagcgagcacagctggattggcttactcagcctaaagtcagtggagcggtctgatgctggcctgtactggtgccaggtgaaggatggggaggaaaccaagatctctcagtcagtatggctcactgtcgaaggtgtgccattcttcacagtggaaccaaaagatctggcggtgccacccaatgccccttttcagctgtcttgtgaggctgtgggtcctccagaacccgtaaccatttactggtggagaggactcactaaagttgggggacctgctccctctccctctgttttaaatgtgacaggagtgacccagcgcacagagttttcttgtgaagcccgcaacataaaaggcctggccacttcccgaccagccattgttcgccttcaagcaccgcctgcagctcctttcaacaccacagtaacaacgatctccagctacaacgctagcgtggcctgggtgccaggtgctgacggcctagctctgctgcattcctgtactgtacaggtggcacacgccccaggagaatgggaggcccttgctgttgtggttcctgtgccaccttttacctgcctgcttcggaacttggcccctgccaccaactacagccttagggtgcgctgtgccaatgccttgggcccttctccctacggcgactgggtgccctttcagacaaagggcctagcgccagccagagctcctcagaatttccatgccattcgtaccgactcaggccttatcctggaatgggaagaagtgattcctgaggaccctggggaaggccccctaggaccttataagctgtcctgggtccaagaaaatggaacccaggatgagctgatggtggaagggaccagggccaatctgaccgactgggatccccagaaggacctgattttgcgtgtgtgtgcctccaatgcaattggtgatgggccctggagtcagccactggtggtgtcttctcatgaccatgcagggaggcagggccctccccacagccgcacatcctgggtgcctgtggtcctgggcgtgctcaccgccctgatcacagctgctgccttggccctcatcctgcttcggaagagacgcaaggagacgcgtttcgggcaagcctttgacagtgtcatggcccgaggggagccagctgtacacttccgggcagcccgatctttcaatcgagaaaggcctgaacgcattgaggccacattggatagcctgggcatcagcgatgaattgaaggaaaagctggaggatgtcctcattccagagcagcagttcaccctcggtcggatgttgggcaaaggagagtttggatcagtgcgggaagcccagctaaagcaggaagatggctccttcgtgaaagtggcagtgaagatgctgaaagctgacatcattgcctcaagcgacatagaagagttcctccgggaagcagcttgcatgaaggagtttgaccatccacacgtggccaagcttgttggggtgagcctccggagcagggctaaaggtcgtctccccattcccatggtcatcctgcccttcatgaaacatggagacttgcacgcctttctgctcgcctcccgaatcggggagaacccttttaacctgcccctgcagaccctggtccggttcatggtggacattgcctgtggcatggagtacctgagctcccggaacttcatccaccgagacctagcagctcggaattgcatgctggccgaggacatgacagtgtgtgtggctgattttggactctctcggaaaatctatagcggggactattatcgtcagggctgtgcctccaaattgcccgtcaagtggctggccctggagagcttggctgacaacttgtatactgtacacagtgatgtgtgggccttcggggtgaccatgtgggagatcatgactcgtgggcagacgccatatgctggcattgaaaatgctgagatttacaactacctcatcggcgggaaccgcctgaagcagcctccggagtgcatggaggaagtgtatgatctcatgtaccagtgctggagcgccgaccccaagcagcgcccaagcttcacgtgtctgcgaatggaactggagaacattctgggccacctgtctgtgctgtccaccagccaggaccccttgtacatcaacattgagagagctgagcagcctactgagagtggcagccctgagctgcactgtggagagcgatccagcagcgaggcaggggacggcagtggcgtgggggcagtaggtggcatccccagtgactctcggtacatcttcagccccggagggctatccgagtcaccagggcagctggagcagcagccagaaagccccctcaatgagaaccagaggctgttgttgctgcagcaagggctactgcctcacagtagctgttaaccctcaggcagaggaaagttggggcccctggctctgctgaccactgtgctgcctgactaggcccagtctgatcacagcccaggcagcaaggtatggaggctcctgtggtagccctcccaagctgtgctggcgcctggacggaccaaattgcccaatcccagttcttcctgcagccgctctggccagcctggcatcagttcaggccttggcttagaggaggtgagccagagctggttgcctgaatgcaggcagctggcaggaggggagggtggctatgtttccatgggtaccatgggtgtggatggcagtaagggagggtagcaacagccctgtgggcccctaccctcctggctgagctgctcctactttagtgcatgcttggagccgcctgcagcctggaactcagcactgcccaccacacttgggccgaaatgccaggtttgcccctcttaagtcacaaagagatgtccatgtattgttcccttttaggtgatgattaggaagggattggcacacttgggtccctaagccctatggcaggaaatggtgggatattctcaggtctgaatcctcatcatcttcctgattccccaccctgcaaaggcctggaactggctgtggggctctgaggcatgctgaaggacaaaagattacagagatccgacttcaaaaggcagggtctgagtctggcaggtggagaggtgctaaggggctggcccaggagtcaggcatttcaggacccctccaagcttctacagtctgtctgagcatgctaccaagcccccagataccccaaaactaacagaggcagttttgtctgagcccagccctcccacatgatgacccttaggtctaccctcctctctaaatggacatcctcgtttgtcccaagtctccagagagactactgatggctgatgtgggtaagaaaagttccaggaaccagggctggggtggaaccagggctggggtcgaggcaggctcttgggcaggctcttgctgttaggaacatttctaagctattaagttgctgtttcaaaacaaataaaattgaaacataaaga

SEQ ID NO: 8 - DNA (cDNA), 2550 bp; SINGLE-stranded, LINEAR
gcaggcctgaagctcatgggcgccccagtgaagatgaccgtgtctcaggggcagccagtgaagctcaactgcagcgtggaggggatggaggaccctgacatccactggatgaaggatggcaccgtggtccagaatgcaagccaggtgtccatctccatcagcgagcacagctggattggcttactcagcctaaagtcagtggagcggtctgatgctggcctgtactggtgccaggtgaaggatggggaggaaaccaagatctctcagtcagtatggctcactgtcgaaggtgtgccattcttcacagtggaaccaaaagatctggcggtgccacccaatgccccttttcagctgtcttgtgaggctgtgggtcctccagaacccgtaaccatttactggtggagaggactcactaaagttgggggacctgctccctctccctctgttttaaatgtgacaggagtgacccagcgcacagagttttcttgtgaagcccgcaacataaaaggcctggccacttcccgaccagccattgttcgccttcaagcaccgcctgcagctcctttcaacaccacagtaacaacgatctccagctacaacgctagcgtggcctgggtgccaggtgctgacggcctagctctgctgcattcctgtactgtacaggtggcacacgccccaggagaatgggaggcccttgctgttgtggttcctgtgccaccttttacctgcctgcttcggaacttggcccctgccaccaactacagccttagggtgcgctgtgccaatgccttgggcccttctccctacggcgactgggtgccctttcagacaaagggcctagcgccagccagagctcctcagaatttccatgccattcgtaccgactcaggccttatcctggaatgggaagaagtgattcctgaggaccctggggaaggccccctaggaccttataagctgtcctgggtccaagaaaatggaacccaggatgagctgatggtggaagggaccagggccaatctgaccgactgggatccccagaaggacctgattttgcgtgtgtgtgcctccaatgcaattggtgatgggccctggagtcagccactggtggtgtcttctcatgaccatgcagggaggcagggccctccccacagccgcacatcctgggtgcctgtggtcctgggcgtgctcaccgccctgatcacagctgctgccttggccctcatcctgcttcggaagagacgcaaggagacgcgtttcgggcaagcctttgacagtgtcatggcccgaggggagccagctgtacacttccgggcagcccgatctttcaatcgagaaaggcctgaacgcattgaggccacattggatagcctgggcatcagcgatgaattgaaggaaaagctggaggatgtcctcattccagagcagcagttcaccctcggtcggatgttgggcaaaggagagtttggatcagtgcgggaagcccagctaaagcaggaagatggctccttcgtgaaagtggcagtgaagatgctgaaagctgacatcattgcctcaagcgacatagaagagttcctccgggaagcagcttgcatgaaggagtttgaccatccacacgtggccaagcttgttggggtgagcctccggagcagggctaaaggtcgtctccccattcccatggtcatcctgcccttcatgaaacatggagacttgcacgcctttctgctcgcctcccgaatcggggagaacccttttaacctgcccctgcagaccctggtccggttcatggtggacattgcctgtggcatggagtacctgagctcccggaacttcatccaccgagacctagcagctcggaattgcatgctggccgaggacatgacagtgtgtgtggctgattttggactctctcggaaaatctatagcggggactattatcgtcagggctgtgcctccaaattgcccgtcaagtggctggccctggagagcttggctgacaacttgtatactgtacacagtgatgtgtgggccttcggggtgaccatgtgggagatcatgactcgtgggcagacgccatatgctggcattgaaaatgctgagatttacaactacctcatcggcgggaaccgcctgaagcagcctccggagtgcatggaggaagtgtatgatctcatgtaccagtgctggagcgccgaccccaagcagcgcccaagcttcacgtgtctgcgaatggaactggagaacattctgggccacctgtctgtgctgtccaccagccaggaccccttgtacatcaacattgagagagctgagcagcctactgagagtggcagccctgagctgcactgtggagagcgatccagcagcgaggcaggggacggcagtggcgtgggggcagtaggtggcatccccagtgactctcggtacatcttcagccccggagggctatccgagtcaccagggcagctggagcagcagccagaaagccccctcaatgagaaccagaggctgttgttgctgcagcaagggctactgcctcacagtagctgt

SEQ ID NO: 9 - DNA (cDNA), 4364 bp; SINGLE-stranded, LINEAR
cattagatctttacatgaaagtaaaatttataagatttctagaaagtcaaaagatgataactatttcttaggatactaaaagcactcacattatagaaaaaaaatcagttaactatactccacaaacattaaaggctccctataaaaaaacatttttaataggcaagccacagaaagggcaaatattaatagtttgcaatacatatgtatgaaaaggaattgaatctagaatatttaacaaagctttacaactcaaaaaatacaaagaaaatatttttcttccaattggcaaattacttaaacagaaccttcacaaaagaagataagaatgtttaataaacatttgaagccataataatgacatcattagccatgatggaaatgcaaatttaagtaccacttcacatccacaagaaaaagataaaaataaaaggactgagctcaccaaacattggtgaggatgtggtaatactgaaattcttgtaccgtgctcctgagggtataacatattacaggatttttttgaaaactagtggttccttataaacttaatgccctggcaacctcacacctatttacttaagaatgaaagggccccgccctcctccctcctcgctcgcgggccgggcccggcatggtgcggcgtcgccgccgatggcgctgaggcggagcatggggcggccggggctcccgccgctgccgctgccgccgccaccgcggctcgggctgctgctggcggagtccgccgccgcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgctgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgcacatcctgggtacctgtggtccttggtgtgctaacggccctggtgacggctgctgccctggccctcatcctgcttcgaaagagacggaaagagacgcggtttgggcaagcctttgacagtgtcatggcccggggagagccagccgttcacttccgggcagcccggtccttcaatcgagaaaggcccgagcgcatcgaggccacattggacagcttgggcatcagcgatgaactaaaggaaaaactggaggatgtgctcatcccagagcagcagttcaccctgggccggatgttgggcaaaggagagtttggttcagtgcgggaggcccagctgaagcaagaggatggctcctttgtgaaagtggctgtgaagatgctgaaagctgacatcattgcctcaagcgacattgaagagttcctcagggaagcagcttgcatgaaggagtttgaccatccacacgtggccaaacttgttggggtaagcctccggagcagggctaaaggccgtctccccatccccatggtcatcttgcccttcatgaagcatggggacctgcatgccttcctgctcgcctcccggattggggagaacccctttaacctacccctccagaccctgatccggttcatggtggacattgcctgcggcatggagtacctgagctctcggaacttcatccaccgagacctggctgctcggaattgcatgctggcagaggacatgacagtgtgtgtggctgacttcggactctcccggaagatctacagtggggactactatcgtcaaggctgtgcctccaaactgcctgtcaagtggctggccctggagagcctggccgacaacctgtatactgtgcagagtgacgtgtgggcgttcggggtgaccatgtgggagatcatgacacgtgggcagacgccatatgctggcatcgaaaacgctgagatttacaactacctcattggcgggaaccgcctgaaacagcctccggagtgtatggaggacgtgtatgatctcatgtaccagtgctggagtgctgaccccaagcagcgcccgagctttacttgtctgcgaatggaactggagaacatcttgggccagctgtctgtgctatctgccagccaggaccccttatacatcaacatcgagagagctgaggagcccactgtgggaggcagcctggagctacctggcagggatcagccctacagtggggctggggatggcagtggcatgggggcagtgggtggcactcccagtgactgtcggtacatactcacccccggagggctggctgagcagccagggcaggcagagcaccagccagagagtcccctcaatgagacacagaggcttttgctgctgcagcaagggctactgccacacagtagctgttagcccacaggcagagggcatcggggccatttggccggctctggtggccactgagctggctgactaagccccgtctgaccccagcccagacagcaaggtgtggaggctcctgtggtagtcctcccaagctgtgctgggaagcccggactgaccaaatcacccaatcccagttcttcctgcaaccactctgtggccagcctggcatcagtttaggccttggcttgatggaagtgggccagtcctggttgtctgaacccaggcagctggcaggagtggggtggttatgtttccatggttaccatgggtgtggatggcagtgtggggagggcaggtccagctctgtgggccctaccctcctgctgagctgcccctgctgcttaagtgcatgcattgagctgcctccagcctggtggcccagctattaccacacttggggtttaaatatccaggtgtgcccctccaagtcagaaagagatgtccttgtaatattcccttttaggtgagggttggtaaggggttggtatctcaggtctgaatcttcaccatctttctgattccgcaccctgcctacgccaggagaagttgaggggagcatgcttccctgcagctgaccgggtcacacaaaggcatgctggagtacccagcctatcaggtgcccctcttccaaaggcagcgtgccgagccagcaagaggaaggggtgctgtgaggcttgcccaggagcaagtgaggccggagaggagttcaggaacccttctccatacccacaatctgagcacgctaccaaatctcaaaatatcctaagactaacaaaggcagctgtgtctgagcccaacccttctaaacggtgacctttagtgccaacttcccctctaactggacagcctcttctgtcccaagtctccagagagaaatcaggcctgatgagggggaattcctggaacctggaccccagccttggtgggggagcctctggaatgcatggggcgggtcctagctgttagggacatttccaagctgttagttgctgtttaaaatagaaataaaattgaagactaaagaccta

SEQ ID NO: 10 - DNA (cDNA), 2550 bp; SINGLE-stranded, LINEAR
gcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgctgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgcacatcctgggtacctgtggtccttggtgtgctaacggccctggtgacggctgctgccctggccctcatcctgcttcgaaagagacggaaagagacgcggtttgggcaagcctttgacagtgtcatggcccggggagagccagccgttcacttccgggcagcccggtccttcaatcgagaaaggcccgagcgcatcgaggccacattggacagcttgggcatcagcgatgaactaaaggaaaaactggaggatgtgctcatcccagagcagcagttcaccctgggccggatgttgggcaaaggagagtttggttcagtgcgggaggcccagctgaagcaagaggatggctcctttgtgaaagtggctgtgaagatgctgaaagctgacatcattgcctcaagcgacattgaagagttcctcagggaagcagcttgcatgaaggagtttgaccatccacacgtggccaaacttgttggggtaagcctccggagcagggctaaaggccgtctccccatccccatggtcatcttgcccttcatgaagcatggggacctgcatgccttcctgctcgcctcccggattggggagaacccctttaacctacccctccagaccctgatccggttcatggtggacattgcctgcggcatggagtacctgagctctcggaacttcatccaccgagacctggctgctcggaattgcatgctggcagaggacatgacagtgtgtgtggctgacttcggactctcccggaagatctacagtggggactactatcgtcaaggctgtgcctccaaactgcctgtcaagtggctggccctggagagcctggccgacaacctgtatactgtgcagagtgacgtgtgggcgttcggggtgaccatgtgggagatcatgacacgtgggcagacgccatatgctggcatcgaaaacgctgagatttacaactacctcattggcgggaaccgcctgaaacagcctccggagtgtatggaggacgtgtatgatctcatgtaccagtgctggagtgctgaccccaagcagcgcccgagctttacttgtctgcgaatggaactggagaacatcttgggccagctgtctgtgctatctgccagccaggaccccttatacatcaacatcgagagagctgaggagcccactgtgggaggcagcctggagctacctggcagggatcagccctacagtggggctggggatggcagtggcatgggggcagtgggtggcactcccagtgactgtcggtacatactcacccccggagggctggctgagcagccagggcaggcagagcaccagccagagagtcccctcaatgagacacagaggcttttgctgctgcagcaagggctactgccacacagtagctgt

SEQ ID NO: 11 - DNA (cDNA), 1158 bp; SINGLE-stranded, LINEAR
gcaggcctgaagctcatgggcgccccagtgaagatgaccgtgtctcaggggcagccagtgaagctcaactgcagcgtggaggggatggaggaccctgacatccactggatgaaggatggcaccgtggtccagaatgcaagccaggtgtccatctccatcagcgagcacagctggattggcttactcagcctaaagtcagtggagcggtctgatgctggcctgtactggtgccaggtgaaggatggggaggaaaccaagatctctcagtcagtatggctcactgtcgaaggtgtgccattcttcacagtggaaccaaaagatctggcggtgccacccaatgccccttttcagctgtcttgtgaggctgtgggtcctccagaacccgtaaccatttactggtggagaggactcactaaagttgggggacctgctccctctccctctgttttaaatgtgacaggagtgacccagcgcacagagttttcttgtgaagcccgcaacataaaaggcctggccacttcccgaccagccattgttcgccttcaagcaccgcctgcagctcctttcaacaccacagtaacaacgatctccagctacaacgctagcgtggcctgggtgccaggtgctgacggcctagctctgctgcattcctgtactgtacaggtggcacacgccccaggagaatgggaggcccttgctgttgtggttcctgtgccaccttttacctgcctgcttcggaacttggcccctgccaccaactacagccttagggtgcgctgtgccaatgccttgggcccttctccctacggcgactgggtgccctttcagacaaagggcctagcgccagccagagctcctcagaatttccatgccattcgtaccgactcaggccttatcctggaatgggaagaagtgattcctgaggaccctggggaaggccccctaggaccttataagctgtcctgggtccaagaaaatggaacccaggatgagctgatggtggaagggaccagggccaatctgaccgactgggatccccagaaggacctgattttgcgtgtgtgtgcctccaatgcaattggtgatgggccctggagtcagccactggtggtgtcttctcatgaccatgcagggaggcagggccctccccacagccgc

SEQ ID NO: 12 - DNA (cDNA), 1158 bp; SINGLE-stranded, LINEAR
gcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgctgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgc

SEQ ID NO: 13 - DNA (cDNA), 19 bp; SINGLE-stranded, LINEAR
tggatggcagtaagggagg

SEQ ID NO: 14 - DNA (cDNA), 20 bp; SINGLE-stranded, LINEAR
cttaagaggggcaaacctgg

SEQ ID NO: 15 - DNA (cDNA), 21 bp; SINGLE-stranded, LINEAR
gcttagaggaggtgagccaga

SEQ ID NO: 16 - DNA (cDNA), 18 bp; SINGLE-stranded, LINEAR
tgggcagtgctgagttcc

(19) United States
(12) Patent Application Publication
(10) Pub. No.: US 2002/0137168 A1
(43) Pub. Date: Sep. 26, 2002

(54) Developmental tyrosine kinases and their ligands

(75) Inventors: Philip S. Crosier, Auckland (NZ); Kathryn E. Crosier, Auckland (NZ)

Correspondence Address: NIXON & VANDERHYE P.C., 8th Floor, 1100 North Glebe Road, Arlington, VA, 22201-4714, US

(73) Assignee: Auckland Uniservices Limited

(21) Appl. No.: 09/985,675

(22) Filed: Nov. 5, 2001

Related U.S. Application Data
(63) Continuation of application No. 08/805,180, filed on Feb. 26, 1997, now abandoned.
Application No. 08/805,180 is a continuation of application No. 08/505,241, filed on Aug. 16, 1995, now abandoned.

(30) Foreign Application Priority Data
Feb. 16, 1993 (NZ) 245917
Feb. 16, 1994 (US) PCT/NZ94/00009

(51) Int. Cl. C12N 9/12; C07H 21/04; C12P 21/02; C12N 5/06

(52) U.S. Cl. 435/194; 435/69.1; 435/325; 435/320.1; 536/23.2

(57) ABSTRACT

The invention relates to mammalian receptor tyrosine kinases designated developmental tyrosine kinases (Dtks). Dtks are expressed in multipotential haematopoietic cells, in embryonic stem cells, in brain tissue and in testis, but are not expressed mature lineage-restricted haematopoetic cells.

The invention provides full-sequence Dtks as well as extracellular receptor domains of such Dtks. The invention further provides nucleic acid molecules encoding such Dtks, vectors containing DNA encoding such Dtks, ligands which bind to such Dtks, and methods of therapeutic and/or prophylactic treatment employing either the ligands or extracellular receptor domains.

### FIELD OF THE INVENTION

The present invention generally relates to protein tyrosine kinase receptors widely expressed by early cells of the haematopoietic system, by cells of the neuronal system in brain tissue, and in testis, ligands for such receptors and nucleic acid molecules encoding such receptors.

### BACKGROUND OF THE INVENTION

There are several parallels between the development of the haematopoietic and neuronal systems. In particular, the presence of regulatory protein Molecules termed growth factors which recognise and bind to specific cell membrane receptors is a common feature of these two systems. It is possible that shared families of receptors exist that are expressed in both early haematopoietic and neuronal stem cells. In turn, there may be a family of proteins which bind these receptors and function as stem cell growth factors.

The current view of vertebrate haematopoietic ontogeny holds that a succession of pluripotential stem cell migrations originate in the yolk sac blood islands, initially invade the hepatic rudiment, and then the spleen and bone marrow. From the bone marrow, a limited number of multipotential stem cells are laid down during embryogenesis that give rise to a much larger population of developmentally restricted progenitor cells, and ultimately produce the mature cells of at least eight cell lineages. The cells of these lineages are classified as red and white blood cells. The white blood cells contain the mature cells of the lymphoid and myeloid systems. Lymphoid cells contain T and B lymphocytes and are derived from pre-T and pre-B cells, respectively. The myeloid system comprises several cell types known as granulocytes, platelets, monocytes, macrophages, and megakaryocytes. The granulocytes are further divided into neutrophils, eosinophils, basophils and mast cells (see review by Metcalf D. The Molecular Control of Blood Cells , Harvard Univ Press, 1988).

The haematopoietic system functions by precisely controlling the production of cells in the various lineages. Totipotent haematopoietic stem cells have the ability to both self-renew and differentiate. Stem cells undergo a series of differentiation steps leading to increasingly lineage-restricted progenitor cells. The more mature progenitor cells are restricted to production of only one or two lineages. For some time the colony-forming unit-spleen (CFU-S) assay served to operationally define all stem cells. Recent evidence demonstrates heterogeneity within CFU-S, with only a small fraction of CFU-S capable of contributing to long-term repopulation following ablation of the haematopoietic system by irradiation. It is recognised that stem and progenitor populations are not discrete, but represent a continuum of cells from those of high self-renewal capacity and low probability of differentiation to those cells with low self-renewal probability and high commitment to differentiation. When long-term haematopoiesis is investigated at the clonal level, studies have shown that single stem cell clones are sufficient to maintain haematopoiesis over the lifetime of an animal.

The development of the mammalian embryo is governed by interactions between different embryonic cell populations. This process is manifest at the cellular level in the precise temporal and spatial control of proliferation, differentiation and migration. The coordination of these processes may be achieved in part by the action of a family of regulatory molecules termed growth factors. Growth factors can evoke diverse responses in different cell types and may interact with one another synergistically or antagonistically. Their action is complex and most of our current understanding results from in vitro experiments. In most instances, haematopoietic growth factor actions defined in vitro have been confirmed in vivo. In haematopoiesis, some growth factors are lineage-restricted in their action. These include erythropoietin that acts predominantly on red cell development, and granulocyte colony-stimulating factor that's predominant action is on granulocytes. At the other end of the spectrum is interleukin-3 which can act on several target cells such as granulocyte-macrophage progenitors, eosinophils, megakaryocytes, erythroid cells and mast cells. There are no known growth factors that function exclusively on haematopoietic stem cells.

The ligand for c-kit, termed stem cell factor, kit ligand or mast cell growth factor is the product of the Steel (SI) locus in mice. The factor acts either alone or synergistically with several known growth factors on primitive stem cells. It is believed that this factor is essential for the development of early haematopoietic stem cells, and cells of the erythroid and mast cell lineages.

The stem cell compartment may be viewed as a finely tuned balance between the action of inhibitors and the stimulatory role of cytokines. As with other stem cell systems, haematopoietic stem cells are distributed in a defined spatial manner within adult bones and not in a random, homogeneous mixture of interacting cell types. A concept that underlies the regulation of haematopoietic stem cell development is that these cells reside within a specialised microenvironment, where the regulatory signals act locally. Stromal cells constitute the bone marrow microenvironment.

Embryonic stem cells are permanent cell lines established directly from the inner cell mass of the preimplantation mouse embryo. They retain the ability to participate in normal embryonic development and, following introduction into the blastocyst, generate chimaeric animals that are mosaic in all tissues. Embryonic stem cells are increasingly being used as cellular vectors for experimentally manipulating the mouse genome.

Doetschman has demonstrated that embryonic stem cells can generate primitive erythroid cells in culture (Doetschman et. al. J. Embryol. Exa. Morphol. 87, 27-45, (1985)). This result was achieved by inducing embryonic stem cells to form cystic embryoid bodies in the presence of preselected batches of human cord serum.

In addition to haematopoietic cell development, it has been noted that neurons also arise in differentiating embryonic stem cells. Haematopoietic differentiation in this system occurred infrequently, slowly and was not synchronized. Recently a modified system enabling the differentiation of embryonic stem cells in methylcellulose into multiple haematopoietic lineages has been described by Wiles and Keller (Development 111, 259-267, (1991)). Using this approach, macrophages, neutrophils, erythroid cells and mast cells develop in a synchronous manner with high frequency in the absence of human cord serum. The development of haematopoiesis from embryonic stem cells in methylcellulose cultures parallels the onset of haematopoiesis in the developing mouse embryo.

An important objective in the field of developmental biology is the identification of genes, the products of which mediate regulatory signals required during embryogenesis. There is compelling evidence that genes encoding receptor tyrosine kinases (RTKs) are involved in early development in vertebrates. The general family of protein tyrosine kinases can be recognised by the presence of several conserved amino acid regions in the catalytic domain. These conserved regions have been summarised by Hanks et al (Science 241, 42-52, (1988)) and by Wilks et al (Proc. Natl. Acad. Sci. USA 86, 1603-1607, (1989)). The receptor for macrophage colony-stimulating factor c-fms, which is important in myeloid cell differentiation and placental development is an RTK. The mouse developmental mutation W has been shown to involve an RTK. The W locus encodes the c-kit RTK and affects the proliferative and/or migratory properties of primordial germ cells, melanoblasts and haematopoietic stem cells. A recently described RTK termed flk-2, which is related to c-kit, has been isolated using the polymerase chain reaction (PCR) with oligonucleotides to conserved kinase domain motifs. Messenger RNA transcripts for flk-2 are expressed in populations enriched for stem cells and primitive uncommitted progenitor cells, and are absent in mature haematopoietic cells (see Matthews et al. Cell 65, 1143-1152, (1991)).

Additional receptor tyrosine kinases expressed on pluripotential haematopoietic stem cells are needed to facilitate the in vitro growth of stem cells. The nucleic acid molecules that encode receptor tyrosine kinases expressed by pluripotential stem cells are needed to produce recombinant receptors and ligands.

In vertebrate development, the cells whose descendants give rise to the nervous system are first identified as the neural ectoderm. This forms a tube-like structure beneath the surface of the ectoderm. Following closure of the neural tube some precursor cells detach from the apical neural tube and form a transient structure called the neural crest. These cells rapidly disperse into the embryo along complex migratory pathways. The proliferating neural crest cells also invade developing tissues such as the skin, gut, and the adrenal gland to form differentiated cell populations within these tissues; eg. melanocytes, enteric neurons and adrenal medullary chromaffin cells.

The diversity of cell types derived from the neural crest poses the problem of how uncommitted embryonic cells acquire particular developmental fates. There are strong parallels between neural crest cell lineage diversification and the process of haematopoiesis. It has been proposed that the earliest neural crest cells should be multipotent and maybe capable of self renewal. Secondly, it should be possible to identify committed progenitors that proliferate symmetrically and are restricted to distinct sublineages and thirdly, there should exist factors which influence the proliferation and/or differentiation of specific types of progenitors (see Anderson Neuron 3, 1-12, (1989)).

Soluble proteins variously termed neurotrophic, growth, and neuronal differentiation factors have been identified that influence the developmental growth, maintenance of function, and plasticity of neuronal populations. These factors have been implicated in the proliferation and differentiation of neurons during embryonic development and in their growth and survival in the adult nervous system. There are a growing number of neurotrophic factors, including nerve growth factor, brain-derived neurotrophic factor, neurotrophin-3 and neurotrophin-4. These molecules constitute a closely related family sharing at least 60% amino acid identity. If the parallel to the haematopoietic system is extended, the range and complexity of cells derived from the neural crest implies that there will be a large number of protein regulators which control this system.

Two different types of receptors have been demonstrated for neurotrophins. One group of these receptors are transmembrane glycoproteins with tyrosine kinase activity encoded by members of the trk protooncogene family. It would therefore be important to isolate additional receptor tyrosine kinases from developing systems such as embryonic stem cells which contain neurons. Ligands for such receptors are required to act inter alia as neurotrophic factors. Nucleic acid molecules encoding the receptors and ligands are needed to produce recombinant receptors and ligands.

It is the object of the present invention to go some way towards fulfilling the above objectives or at least to provide the public with a useful choice.

### SUMMARY OF THE INVENTION

The present invention has a number of aspects. In a first aspect, the invention provides a mammalian receptor tyrosine kinase which is a developmental tyrosine kinase (Dtk) and which is expressed in multipotential haematopoietic cells, in embryonic stem cells, in brain tissue and in testis, but which is not expressed in mature lineage-restricted haematopoietic cells.

In a further aspect, the invention provides an extracellular receptor domain of a receptor tyrosine kinase as defined above. In preferred embodiments, this extracellular receptor domain can be bound to a support, or can be in a soluble form.

In still a further aspect, the invention provides a nucleic acid molecule encoding a receptor tyrosine kinase or extracellular receptor domain as defined above. This nucleic acid molecule is preferably DNA.

In yet a further aspect, the invention provides a vector including a DNA molecule as defined above.

In still a further aspect, the invention provides a method of producing a receptor tyrosine kinase comprising the steps of:

(a) culturing a host cell which has been transformed or transfected with a vector as defined above to express the encoded receptor tyrosine kinase or extracellular receptor domain; and

(b) recovering the expressed receptor tyrosine kinase.

As yet an additional aspect, the invention provides a ligand that binds to a receptor tyrosine kinase as defined above.

The ligand can take two forms. In one form, the ligand stimulates the proliferation, differentiation and/or survival of cells which express a receptor tyrosine kinase as defined above (a stimulant ligand).

In the second form, the ligand is antagonistic and at least partially blocks or inhibits the function of a receptor tyrosine kinase as defined above through binding to said receptor (an antagonistic ligand).

In another aspect, the invention provides a method of stimulating the proliferation, differentiation and/or survival of a cell expressing a receptor tyrosine kinase as defined above comprising contacting the cell with a stimulant ligand as defined above.

In yet a further aspect, the invention provides a method of inhibiting the function of a receptor tyrosine kinase as defined above comprising contacting the receptor with an antagonistic ligand as defined above.

In still another aspect, the invention provides a method of treating a disease, syndrome or condition caused or mediated by an excess of a ligand as defined above comprising the step of contacting said excess of said ligand with an effective amount of a receptor tyrosine kinase or an extracellular receptor domain as defined above.

In another aspect, the invention provides a method of extracting a ligand from a medium which may contain said ligand comprising the step of contacting said medium with a receptor tyrosine kinase or with an extracellular receptor domain as defined above.

The invention also provides a method of isolating ligand(s) from a medium which may contain said ligand(s), comprising the steps of:

(a) contacting said medium with an effective amount of a receptor tyrosine kinase or an extracellular domain as defined above;

(b) detecting which ligand(s) bind; and

(c) isolating such bound ligand(s).

While the invention broadly consists in the foregoing, it should be appreciated that it also includes the more specific embodiments detailed in the following description:

### DESCRIPTION OF THE FIGURES

FIG. 1 shows expression of murine Dtk in embryonic stem (ES) cells and embryoid bodies. RNase protection analysis was performed on total RNA (10 μg) from ESD3 ES cells growing in Leukemia Inhibitory Factor (LIF) (day 0), or from ES cells maintained in the absence of LIF that were differentiating and developing into cystic embryoid bodies (days 2 to 18). As a control tRNA (10 μg) was also used. The markers were pBR322 digested with Msp I. The size of the free murine Dtk probe was 229 nt. A fully protected fragment representing the presence of murine Dtk transcripts was 187 nt in length. The free B-actin protected fragment is shown in each lane as an RNA loading control.

FIG. 2 shows expression of murine Dtk in embryonic mouse tissues. RNase protection analysis-was performed on total RNA (10 μg) isolated from E14.5 embryonic tissues of the C57BL/6J mouse strain. Details of the markers, probes and controls are as described for FIG. 1.

FIGS. 3 and 4 show expression of murine Dtk in adult mouse tissues. RNase protection analysis was performed on total RNA (10 μg) isolated from the various tissues of adult C57BL/6J mice. Details of the markers, probes and controls are as described for FIG. 1.

FIG. 5 shows expression of murine Dtk in murine cell lines. The most abundant expression is in the multipotential cell lines FDC-P1 and DA2, and the mast cell line P815. The majority of other cell lines art lineage-committed, mature haematopoietic cell lines, which have very limited murine Dtk expression. The NIH 3T3 cell line is derived from embryonic fibroblasts and C2C12 is a myoblast cell line.

FIG. 6 shows the cDNA and amino acid sequence of murine Dtk.

FIG. 7 shows the cDNA and amino acid sequence of human Dtk.

### DETAILED DESCRIPTION OF THE INVENTION

A. Receptors

In a first aspect, this invention provides a mammalian receptor protein tyrosine kinase (PTK). The mammal in which the PTK exists may be any mammal, such as a mouse, rat, rabbit or human.

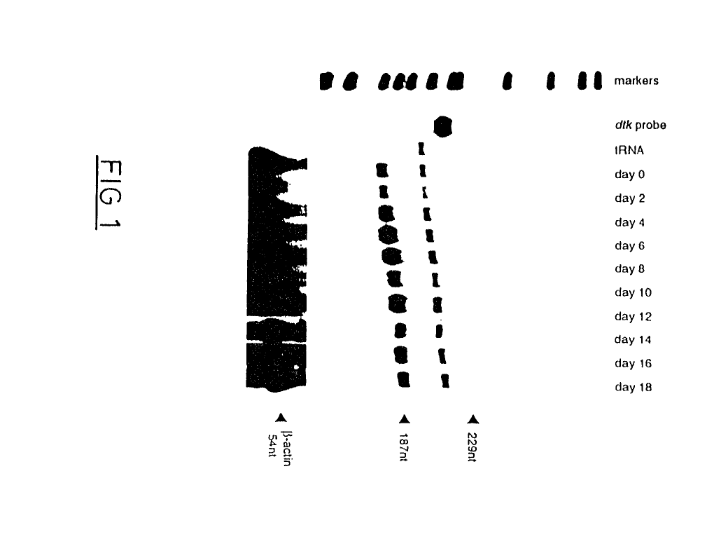

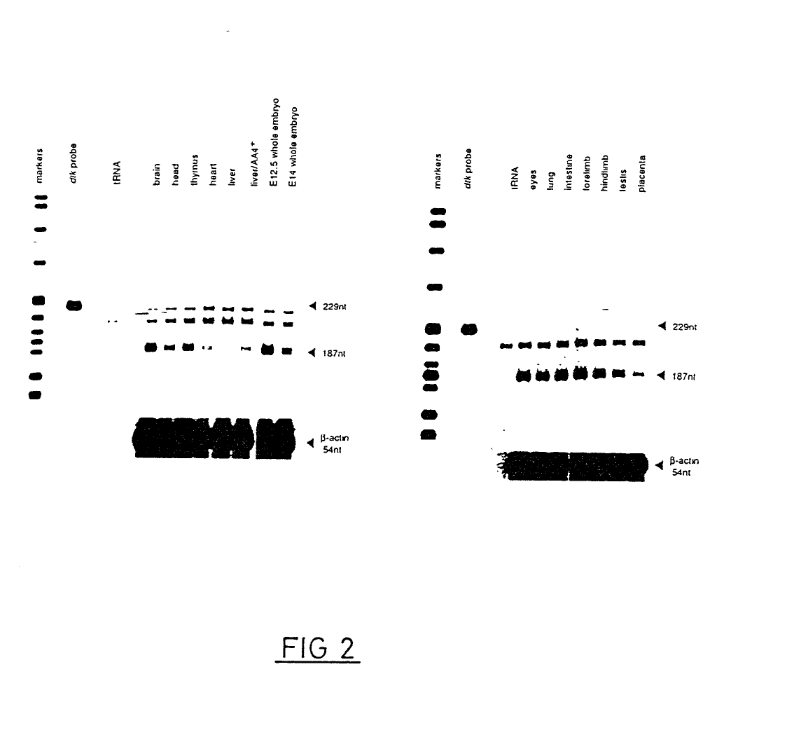

Members of the PTK family are recognised by the conserved amino acid regions in the catalytic domains. Examples of PTK consensus sequences have been provided by Hanks et al. (Science 241 42-52 (1988), especially FIG. 1 starting at page 46) and by Wilks et al. (Proc. Natl. Acad. Sci. USA 86, 1603-1607 (1989), especially FIG. 2 on page 1605).

Hanks et al. identify eleven catalytic subdomains containing FIK consensus residues and sequences. The PTKs of the present invention contain most or all of these consensus residues and sequences.

As indicated above, the PTKs of the invention are receptor PTKs and so are also generally referred to as RTKs. Further, as the applicants believe that the RTKs of the invention are involved in mammalian cell development, they are specifically referred to hereinafter as developmental tyrosine kinases (Dtks).

The Dtks of the invention are transmembrane receptor tyrosine kinases whose extracellular domains contain two immunoglobulin-like motifs followed by two fibronectin-type III repeats. RTKs of this structure (Axl(Ufo,Ark)) are already known (Janssen et al., Oncozene 6, 2113-2120 (1991); O'Bryan et al., Mol. Cell. Biol 11, 5016-5031 (1991); Rescigno et al. Oncogene 6, 1909-1913 (1991); Faust et al. Oncogene 7, 1287-1293 (1992)). The Dtks of the invention are however distinguished from those RTKs having the equivalently structured extracellular domains by their potential function based upon their distribution within the mammalian body.

With regard to this latter feature of the Dtks of the invention, the applicants have conducted experiments to determine the range of cells in which the developmental tyrosine kinases of the invention are expressed. These experiments were specifically performed in relation to murine Dtk but are believed to be illustrative of the expression of all mammalian Dtks of the invention.

A.1 Analysis of Murine Dtk Expression

The expression of murine Dtk in a range of embryonic and adult mouse tissues was analyzed by ribonuclease protection analysis, using a probe that encompassed sequences encoding the membrane-proximal portion of the extracellular domain of the receptor.

Materials and Methods

1. Embryonic Stem Cell Culture

The ESD3 embryonic cell line (Doestschman et al., J. Embryol. Exp. Morphol. 87 27-45 (1985)) was maintained on gelatin-coated dishes in Dulbecco's-modified Eagle's medium (DMEM) with additives according to established procedures (Hogan et al., Cold Spring Harbour Laboratory, 1-332 (1986)), in the presence of LIF. Cystic embryoid bodies were established following collagenase treatment of the ES cells and subsequent suspension culture in bacteriological-grade petri dishes in DMEM with additives in the absence of LIF (Wiles and Keller, Development 111, 259-265 (1991)).

2. Fetal Liver Haematopoietic Stem Cell Enrichment

Low density haematopoietic stem cells were isolated from an E14.5 fetal liver cell suspension using equilibrium density centrifugation on a discontinuous metrizamide gradient according to the method of Visser et al., J. Exp. Med, 59, 1576-1590 (1984). Following this procedure, low density fetal liver cells (p24<1.078 g/cm³) were incubated for 20 minutes on ice in DMEM medium with 5 μg/10⁶ cells of AA4 monoclonal antibody (rat IgG_{2b}; McKeam et al., Proc. Natl. Acad. Sci. USA, 82, 7414-7418 (1985)) and washed twice. This antibody has been shown to recognise the most primitive haematopoietic stem cell in fetal liver (Jordan et al., Cell, 61, 953-963 (1990)). The AA4 labelled cells were then incubated on ice for 20 minutes with magnetic beads conjugated with anti-rat IgG antibody as outlined in the manufacturer's protocol (Advanced Magnetics Corp., Cambridge, Mass.). Following incubation, AA4⁺ cells were positively-selected on a magnet. Stem cell enrichment was assessed by re-labelling the cells with the AA4 antibody, followed by a second layer antibody staining with goat anti-rat fluorescein isothiocyanate and flow cytometric analysis on a FACS 440 (Becton Dickinson, San Jose, Calif.).

3. RNA Analysis

RNase protection analysis was performed by hybridization of 10 μg of total RNA to RNA probes that encoded sequences of murine Dtk and B-actin, overnight at 52° C. RNase digestion was performed with RNase T1 (1.75 μ/ml) and RNase A (35 μg/ml) at 37° C. for one hour. The reaction was stopped with proteinase K (333 μg/ml) and SDS (0.3%). The products were run on a 6% urea/acrylamide gel and the autoradiograph exposed at −70° C. The probe for analysis of Dtk expression was derived from nucleotides 1158 to 1334 of the Dtk sequence, a segment which encodes the membrane-proximal portion of the extracellular domain, and which had been subcloned into pGEM-4Z. In an RNase protection assay, the free probe yielded a 229 nucleotide (nt) band, and Dtk transcripts protected a fragment of 187 nt. A riboprobe was also constructed from a Sal I-Sma I fragment of human β-actin. The length of the free β-actin probe was 132 nt and β-actin transcripts protected a 54 nt fragment.

Results

1. Embryonic Stem Cells

FIG. 1 demonstrates the expression of Dtk transcripts in both totipotent ES cells growing in LIF (termed day 0), and in differentiating cystic embryoid bodies growing in the absence of LIF for up to 18 days. In this developmental system Dtk is expressed almost uniformly from days 0 to 18, indicated by the presence of a protected 187 nt band for each RNA analyzed. The two bands of approximately 220 nt and 210 nt present in lanes for each RNA sample analyzed are also present in the tRNA lane and are regarded as nonspecific. Of considerable interest with regard to the importance of this receptor in mouse development is the demonstration of Dtk expression in totipotent ES cells. The ES cells from which RNA was extracted for day 0 analysis were selected from cultures, following morphological assessment by phase-contrast microscopy to confirm that they were undifferentiated.

2. Embryonic Tissues

Expression of Dtk was detected in total RNA isolated from a wide range of midgestational E14.5 embryonic mouse tissues including the brain, eye, thymus, lung, intestine, forelimb, hindlimb and testis (FIG. 2). There was limited expression in heart and unfractionated liver.

FIG. 2 shows enrichment of Dtk transcripts in E14.5 fetal liver low density AA4⁺ haematopoietic stem cells. Following density-gradient centrifugation and positive selection, the cells used for RNA analysis were greater than 95% AA4⁺, as assessed by flow cytometry (data not shown). Dtk expression was also detected in day 14.5 placenta.

3. Adult Tissues

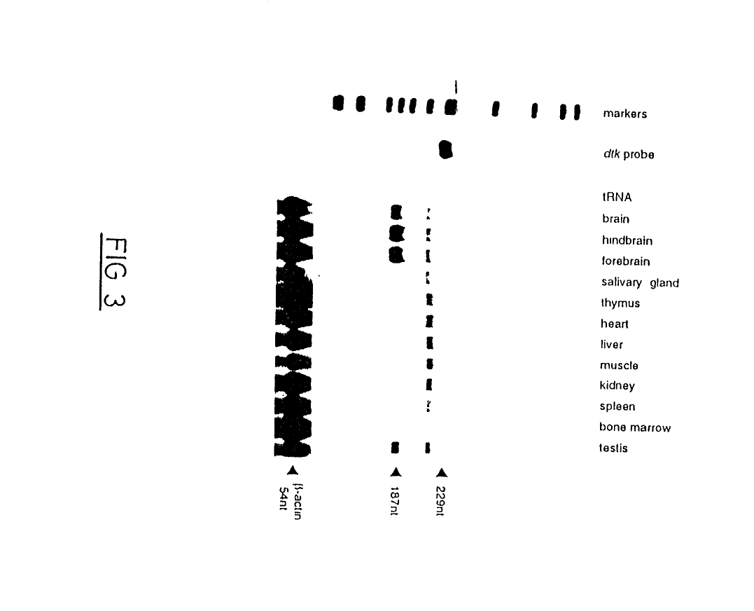

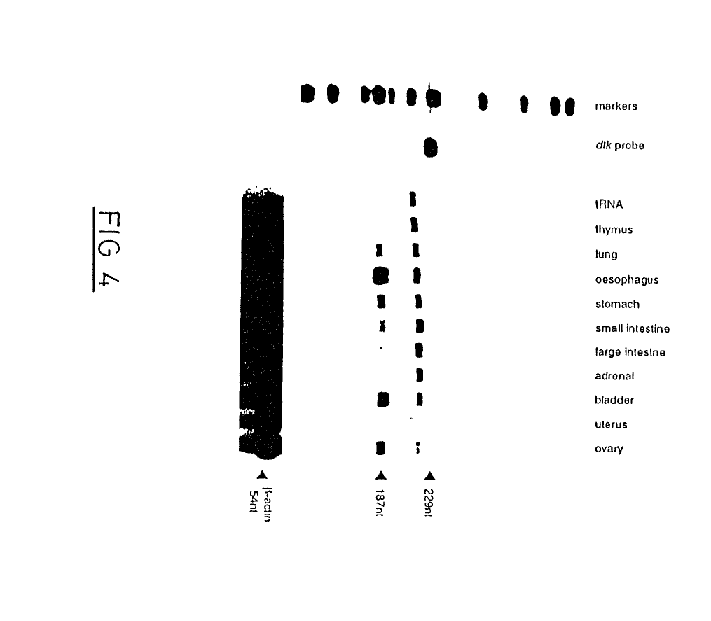

In contrast to the widespread expression of Dtk in embryonic tissues, the pattern of expression in adult tissues becomes restricted (FIGS. 3 and 4). Dtk transcripts were most abundant in brain, esophagus, bladder, testis, and ovary. In brain, expression of Dtk (relative to β-actin) was more abundant in adult than in embryonic tissue. Adult tissues which contained less abundant, but detectable transcripts were lung, and regions of the gastrointestinal tract including the stomach and both the small and large intestine. Tissues in which Dtk transcripts were undetectable or expressed at extremely low levels included the salivary gland, thymus, heart, liver, skeletal muscle, kidney, spleen, bone marrow, adrenal gland and uterus.

4. Murine Cell Lines

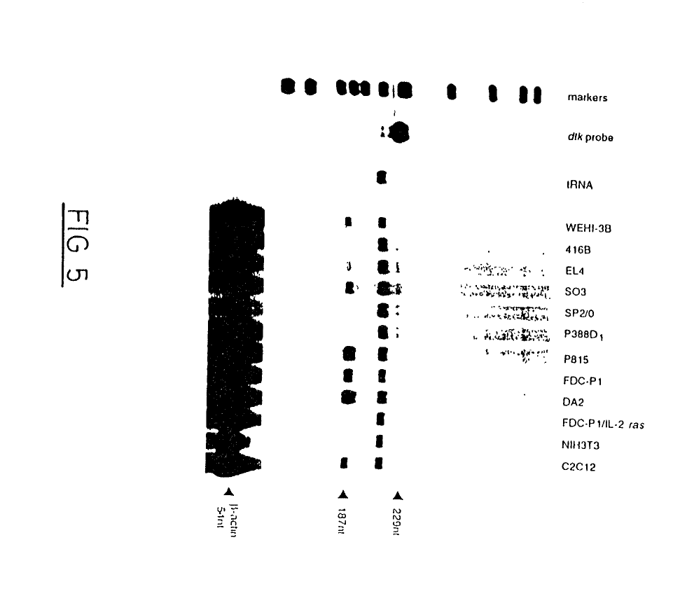

The pattern of expression of Dtk in murine cell lines was analyzed in relation to the following: WEHI-3B, 416B, ELM, SO3, SP2/0, P388D₁, P815, FDC-P1, DA2, FDC-P1/IL-2 ras, NIH3T3 and C2C12. The results are shown in FIG. 5.

As can be seen from FIG. 5, the results are consistent with those above, with the most abundant expression being in the multipotential cell lines FDC-P1 and DA2, and in mast cell line P815. Significant expression is also observed in myoblast cell line C2C12.

In contrast, the remaining cell lines (lineage-restricted mature haematopoietic cell lines) show very limited murine Dtk expression.

From this analysis, the applicants have derived the condition defining the Dtks of the invention—they are expressed in multipotential haematopoietic cells, in totipotent embryonic stem cells, in brain tissue and in testis, but not in mature lineage-restricted haematopoietic cells.

For the purpose of this specification, a multipotential haematopoietic cell is an early haematopoietic cell. Examples of multipotential haematopoietic cells include multipotential factor-dependent cells that have the capacity to proliferate and differentiate into mature haematopoietic cells. In contrast, a mature haematopoietic cell is non self-renewing and has limited ability to give rise to multiple cell lineages. Mature lineage-restricted haematopoietic cells, for the purposes of this specification, are therefore represented by haematopoietic cell lines of the T or B lymphoid lineage or mature myeloid lineages.

The Dtks of the present invention may or may not be expressed in intermediate cells poised between the state of being multipotential and mature.

In terms of brain tissue, the Dtks of the invention are primarily expressed in neuronal cells.

In terms of testis, the Dtks are primarily expressed in the Sertoli cells.

It will of course be appreciated by those persons skilled in this art that the reference to the Dtks of the invention not being expressed in mature lineage-restricted haematopoietic cells is in a biological context and does not mean that there is absolutely no expression of the Dtk in these cells. As is apparent from FIGS. 1 to 5, what is meant by the phrase “not expressed in mature-lineage restricted haematopoietic cells” is that there is no significant expression of the Dtk in the cell, i.e. that expression is either undetectable or at an extremely low level.

The restricted expression of the Dtks of the invention to cells representative of early multipotential cells, with substantial absence of expression in lineage-restricted cells such as T or B lymphocytes, is consistent with this receptor functioning and transducing signals from the microenvironment to the haematopoietic stem cell compartment. The expression of the Dtk in embryonic stem cells and in some fetal tissues such as brain is also consistent with this receptor and its ligand having a functional role in the specification of cell lineages during embryonic development, including neuronal development. Furthermore, the receptor and its ligand is likely to have a role in the maintenance of function and plasticity in neuronal populations or their derivatives. Finally, the expression of the receptor in adult brain is consistent with the receptor and its ligand having a role in the growth and survival of neurons in the adult nervous system.

The embryonic stem cell and haematopoietic multipotential cell line nRNA for Dtk migrates relative to 28S and 18S ribosomal bands on formaldehyde agarose gels at approximately 4.2 Kb. In adult brain tissues, Dtk mRNA migrates at approximately 4.2 Kb.

The Dtks of the invention can usefully be provided in a number of different forms. These include the Dtk itself, the “mature” form of the Dtk, and the extracellular receptor domain of the Dtk.

The “mature” form of the Dtk of the invention is the Dtk less its native amino-terminus leader or signal sequence, whereas the extracellular receptor domain is the Dtk lacking the transmembrane region and catalytic domain.

The extracellular domain may be identified through commonly recognised criteria of extracellular amino acid sequences. The determination of appropriate criteria is known to those skilled in the art, and has been described, for example by Hopp et al., Proc. Natl. Acad. Sci. USA 78, 3824-3828 (1991); Kyte et al., J. Mol. Biol. 157, 105-132 (1982); Emini, J. Virol 55, 836-839 (1985); Jameson et al. CA BIOS 4 181-186 (1988); and Karplus et al. Naturwissenschaften 72, 212-213 (1985). Amino acid domains predicted by these criteria to be surface exposed are characteristic of extracellular domains.

The Dtks of the invention or their extracellular receptor domains may be prepared by methods known in the art. Such methods include protein synthesis from individual amino acids as described by Stuart and Young in “Solid Phase Peptide Synthesis”, Second Edition, Pierce Chemical Company (1984). It is however preferred that the Dtks and/or their extracellular receptor domains be prepared by recombinant methods as will be detailed hereinafter.

A.2 Specific Dtks of the Invention

A.2.1 Murine Dtk

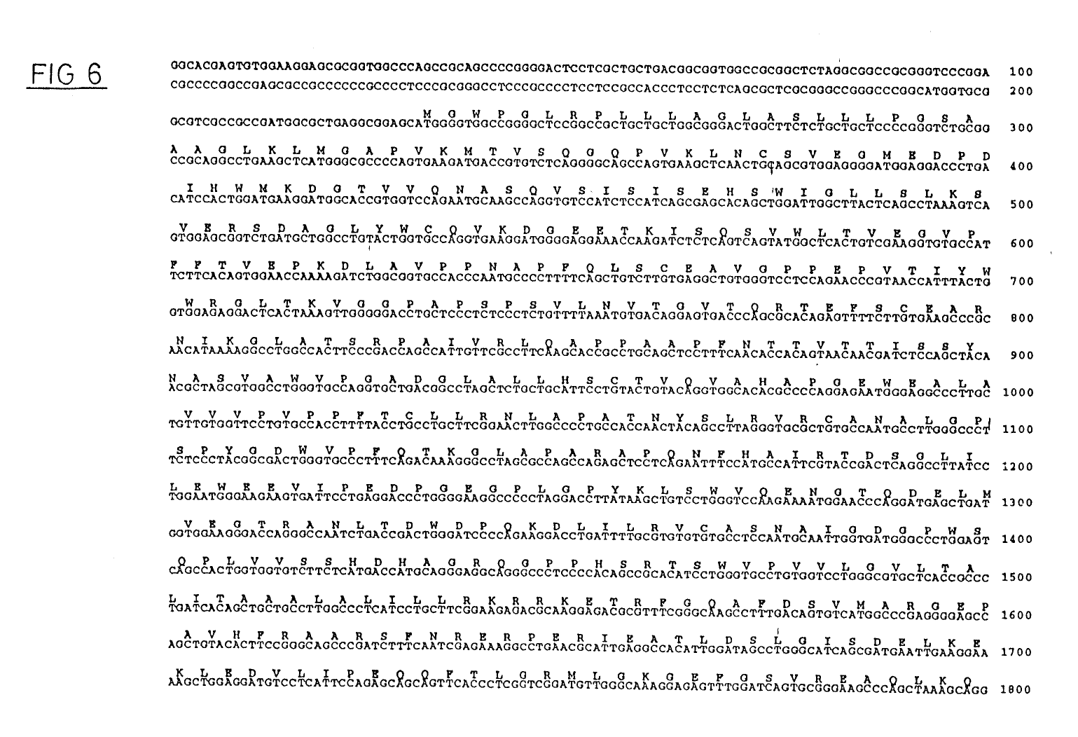

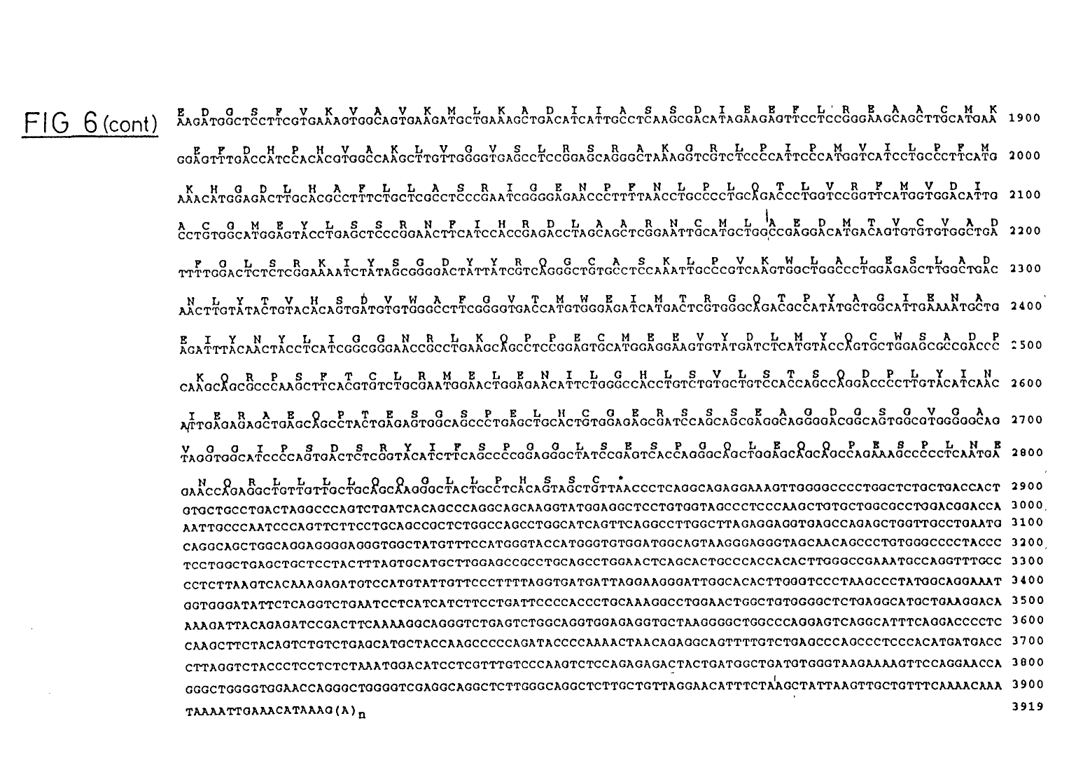

As is indicated above, a first Dtk of the invention, murine Dtk, has been identified in certain tissues of the mouse. Murine Dtk generally has the nucleic acid and deduced amino acid sequence shown in FIG. 6. FIG. 6 represents individual amino acid residues as single letters as follows:

| | Three-letter | One-letter |
|---|---|---|
| Amino Acid | abbreviation | symbol |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gln | Q |
| Glutamic Acid | Glu | E |
| Glutamine or glutamic acid | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Details of the sequence of murine Dtk are as follows.

Sequence Analysis of the Murine Dtk

FIG. 6 shows the 3.919 Kb nucleotide and deduced amino acid sequence for murine Dtk from murine neonatal brain. Within the 5′ region, a potential site for translation initiation (-GGAGCATGGGG-) is found within a good Kozak consensus sequence. The first methionine initiates an open reading frame of 874 amino acids. Using the method of von Heijne Sequence Analysis in Molecular Biology 113-117, San Diego, Academic Press (1987), the signal cleavage site is predicted to be between alanine 24 and alanine 25, which specifies a 24 amino acid hydrophobic leader sequence and a mature receptor tyrosine kinase protein of 850 amino acids. Amino acids AGLK to PHSR form a 386 amino acid extracellular domain. A 25 amino acid hydrophobic region from TSWV to LILL is consistent with that of a transmembrane domain (Fasman and Gilbert, Trends Biochem 15, 89-92 (1990)), while the remaining amino acids ending HSSC comprise the cytoplasmic domain.

The extracellular domain of murine Dtk contains eight consensus sites (NxT or S) for N-linked glycosylation, predicting that the mature Dtk protein is glycosylated. Within the extracellular domain, two repeating protein motifs are identifiable. Using the predictive methods of Williams and Barclay, Ann. Rev. Immunol 6, 381405 (1988), two C-type immunoglobulin (Ig)-like domains are present from amino acids KLMG to GEET (Ig-like domain I) and FFTV to NIKG (Ig-like domain II). The first Ig domain has a structure similar to a C1 domain, while the second Ig domain is more C2-like. Based on the analysis of Petersen et ai, Proc. Natl. Acad. Sci. USA 80, 137-141 (1983), there are two fibronectin type III modules present from amino acids PPAA to PYGD (domain I) and from amino acids PFQT to SHDH (domain II).

Analysis of the 439 amino acid cytoplasmic domain sequence of murine Dtk shows many of the motifs which are highly conserved within the catalytic kinase domain of protein tyrosine kinases (Hanks et al., Science 241, 42-52 (1988)). The motifs GKGEFG and VAVK, which function as the Mg²⁺-ATP binding site (Ullrich and Schlessinger, Cell 61, 203-212 (1990); Cantley et al., Cell 64, 281-302 (1991)), are observed at the start of the kinase domain. Further towards the carboxy-terminus of Dtk other conserved kinase motifs are identifiable, including the motif IHRDLAARN, the DFG triplet motif and the motifs KWLALES and DVWAFG. Alignment of the kinase domain of Dtk with other protein tyrosine kinase domains including that of Ufo, suggests there is a kinase insert region specified by the amino acids RIGENPFN. There are 12 tyrosine residues within the cytoplasmic domain of Dtk, including two residues located near the C-terminus that are nested within sequences that exhibit strong homology to Src homology 2 (SH2) domain bindingasites (Songyang et al., Cell 72, 767-778 (1993)). One of these sequences, EEVYDLM, is a putative binding site for phosphatidylinositol 3-kinase, but lies within the catalytic domain proper and is unlikely to be autophosphorylated. The sequence DPLYINI fulfills criteria for either a Sem5/Grb2 binding site or a phospholipase C-γ binding site (Songyang et al., (1993)) supra, and its position in the C-terminal tail makes it a good candidate for phosphorylation.

In specific aspects, the invention provides murine Dtk, mature murine Dtk and the extracellular receptor domain of murine Dtk.

Murine Dtk has the amino acid sequence given as SEQ ID NO 1.

Mature murine Dtk has the amino acid sequence given as SEQ ID NO 2.

The extracellular receptor domain of murine Dtk has the amino acid sequence given as SEQ ID NO 5.

The invention also includes functional equivalents of murine Dtk, mature murine Dtk and the extracellular receptor domain of murine Dtk as is described hereinafter.

A.2.2 Human Dtk

A second Dtk of the invention has been identified from human tissue. This second receptor is the human homologue of murine Dtk having all of the structural features of murine Dtk.

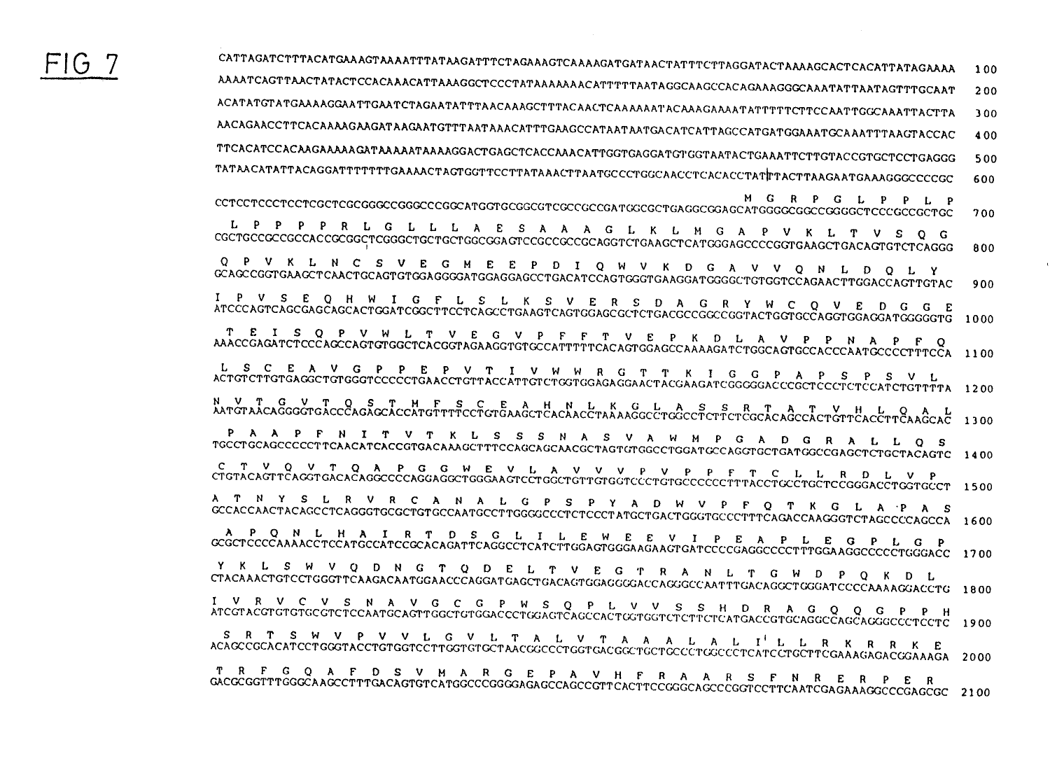

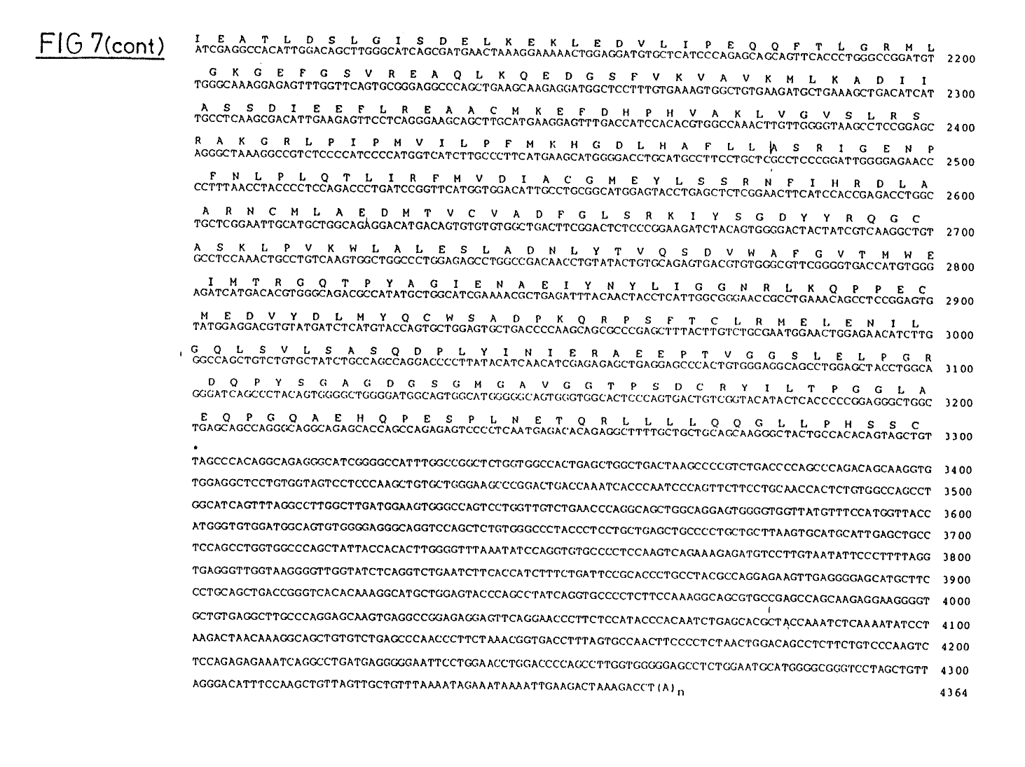

The nucleic acid and deduced amino acid sequence for this receptor tyrosine kinase, hereinafter called “human Dtk”, is shown in FIG. 7. Sequence details are as follows.

Sequence Analysis of the Human Dtk

FIG. 7 shows the 4.364 Kb nucleotide and deduced amino acid sequence for the human Dtk from human fetal brain. The structural features of human Dtk closely parallel those described for murine Dtk. The signal peptide encompasses amino acids MGRP to ESAA. The mature protein extends from residues AGLK to HSSC. Within the mature protein the extracellular domain is defined by residues AGLK to PHSR, the transmembrane domain by residues TSWV to LILL, and the cytoplasmic domain from residues RKRR to HSSC.

The extracellular domain contains two repeating protein motifs made up of two immunoglobulin domains (KLMG to GGET and FFTV to NLKG), followed by two fibronectin type III modules (LPAA to PYAD and PFQT to SHDR). The protein tyrosine kinase domain is encompassed by the amino acids LGKG to RMEL within the cytoplasmic domain. The motifs defined within the murine protein tyrosine kinase domain are also identifiable within the human protein tyrosine kinase domain.

Once again, in its specific aspects the invention provides different forms of the Dtk (human Dtk, “mature” human Dtk and the extracellular receptor domain of human Dtk).

Human Dtk has the amino acid sequence given as SEQ ID NO 3.

Mature human Dtk has the amino acid sequence given as SEQ ID NO 4.

The extracellular receptor domain of human Dtk has the amino acid sequence given as SEQ ID NO 6.

Once again, the invention further includes functional equivalents of human Dtk, mature human Dtk and of the extracellular receptor domain of human DTK.

A.2.3 Other Mammalian Dtks

In addition to the murine and human Dtks described above, the invention includes within its scope Dtks of other mammals. Such Dtks are the homologues of both murine and human Dtk and can be readily identified by those persons skilled in the art with reference to the characterising data given above for murine Dtk and human Dtk.

By way of example, one method for identifying other Dtks of the invention involves the formation of a DNA library from a suitable tissue source (such as brain) obtained from the mammal. This library can then be screened to identify DNA coding for homologues to murine Dtk and human Dtk as will be described in more detail below.

B. Nucleic Acid Molecules Encoding the Dtks of the Invention

In another aspect of this invention, the applicants provide nucleic acid molecules encoding the Dtks. These nucleic acid molecules may be DNA (isolated from nature, synthesised or cDNA) or RNA. Most often, the nucleic acid molecules will be DNA.

B.1 Nucleic Acid Molecules Encoding Murine Dtk and Human Dtk

As indicated above, the nucleic acid sequences for murine Dtk and human Dtk have been determined. In specific aspects, the invention therefore provides nucleic acid molecules (in the form of DNA) as follows:

1. A DNA molecule encoding murine Dtk having the nucleotide sequence given as SEQ ID NO 7.

2. A DNA molecule encoding mature murine Dtk having the nucleotide sequence given as SEQ ID NO 8.

3. A DNA molecule encoding the extracellular receptor domain of murine Dtk having the nucleotide sequence given as SEQ ID NO 11.

4. A DNA molecule encoding human Dtk having the nucleotide sequence given as SEQ ID NO 9.

5. A DNA molecule encoding mature human Dtk having the nucleotide sequence given as SEQ ID NO 10.

6. A DNA molecule encoding the extracellular receptor domain of human Dtk having the nucleotide sequence given as SEQ ID NO 12.

The invention also includes within its scope functional equivalents of these DNA molecules.

B.2 Nucleic Acid Molecules Encoding Dtks of Other Mammals

It will be appreciated that DNA molecules encoding the functional equivalent homologues of murine Dtk and human Dtk from other mammals are also within the scope of the invention. Such DNA molecules can be readily identified using conventional techniques and with reference to the information contained herein characterising murine Dtk and human Dtk.

By way of generic illustration, DNA molecules encoding homologues of murine Dtk and human Dtk in other mammals can be identified by employing the following general steps:

(a) Formation of a cDNA library:

Total mRNA from a suitable tissue source (such as brain) of the mammal is prepared by standard procedures (Ausubel et al, (Eds), “Current Protocols in Molecular Biology” Greene Associates/Wiley Interscience, New York (1990)), and cDNA synthesised. A cDNA library is formed (for example in λZAP II).

(b) Library Screening:

The cDNA library formed as above is screened for the presence of cDNA encoding homologues to murine Dtk and human Dtk. Screening will generally employ a DNA hybridisation or amplification step with the probes or primers being selected based upon the already determined sequences of murine and human Dtk.

Most conveniently, the screening procedure will involve DNA amplification using the polymerase chain reaction (PCR) (Saiki et al Science 239, 487 (1988)) with the PCR primers being selected such that highly conserved regions from within the DNA sequence of murine and human Dtk will be within the amplified PCR product.

(c) DNA Isolation and Sequencing:

Clones from the cDNA library which are identified by screening step (b) as containing cDNA encoding homologues to murine and human Dtk are selected, and the size of the cDNA insert sourced from the brain determined. Such clone(s) including a cDNA insert of the appropriate size to code for the full-length Dtk are selected and the cDNA insert isolated. Each isolated cDNA insert is then sequenced using known procedures (for example, using the standard dideoxy chain-termination method of Sanger et al., Proc. Natl. Acad. Sci. USA 74, 5463-5467 (1977)).

B.3 Genetic Mapping of Murine Dtk and Human Dtk

By way of further characterisation of both murine Dtk and human Dtk, the applicants have performed experiments to establish the chromosomes on which the genes coding for these Dtks are located. Details of these experiments are given below.

Materials and Methods

B.3.1 Fluorescent In Situ Hybridization (FISH)

A partial Sau3A genomic DNA library in λ 2001, prepared from mouse ES cells (Boehm et al., Proc. Natl. Acad. Sci. USA 88, 3927-3931 (1991)), was screened with the 3.525 kb cDNA insert purified from pMo23A using methods previously described (Morris, et al., Blood 76, 1812-1818 (1991)). Of 34 positive clones, two of the most intensely hybridizing, λMo23A-7.1 and λMo23A-8. 1, were selected for FISH studies. The pMo23A plasmid, and DNA isolated from bacteriophage clones λMo23A-7.1 and λMo23A-8.1, were biotinylated by nick-translation using biotin-14-dATP (Bethesda Research Laboratories, Gaithersberg, Md.). Karyotypically normal, 40, XY, mouse metaphase cells were prepared from ES cells in culture using standard procedures. Fluorescent in situ hybridization and detection procedures were essentially as described (Morris et al., Human Genetics 91, 31-36 (1993)), except that mouse Cot 1 DNA (BRL, final concentration 250 ng/μI) was used to suppress repetitive sequences in the two phage DNA probes. Chromosomes were G-banded using DAPI (4′, 6-diamidino-2-phenylindoledihydrochoride, Sigma, St Louis, Mo.) as a counterstain for fluorescence analysis.

B.3.2 Single-strand Conformation Polymorphism (SSCP)

Primer sequences from the 3′ untranslated region of the Dtk cDNA used for genetic mapping were as follows:

| | | |
|---|---|---|
| DtkMap1 | 5′ TGGATGGCAGTAAGGGAGG 3′ | (SEQ ID |
| | | NO: 13) |
| | 5′ CTTAAGAGGGGCAAACCTGG 3′ | (SEQ ID |
| | | NO: 14) |
| DtkMap2 | 5′ GCTTAGAGGAGGTGAGCCAGA 3′ | (SEQ ID |
| | | NO: 15) |
| | 5′ TGGGCAGTGCTGAGTTCC 3′ | (SEQ ID |
| | | NO: 16) |

PCR was performed using standard conditions with the addition of ³²P-labelled dCTP. Specifically, 25,μl reactions were performed in 10 mM-Tris-HCI, 50 mM KCl using 250 ng of genomic DNA, 1 μM of each primer, and 1.4 mM MgCl₂. This was overlaid with oil, denatured at 94° C. for 5 minutes, and transferred to an 80° C. heating block. dNTPs were added to a final concentration of 0.2 mM, including 1.25 μCi of [α-³²P]dCTP (1 μl of a 3000 Ci/mmole stock to 8 reactions). 1.25 units of AmpliTaq DNA polymerase (Perkin-Elmer Cetus) was added and cycling conditions were as follows: 58° C. annealing reaction for 1 minute, 72° C. extension reaction for 2 minutes, and 91° C. denaturation for 1 minute. The cycle was repeated 30 times with a final 72° C. extension reaction for 5 minutes. SSCP analysis was performed by electrophoresing the single-stranded PCR products on a non-denaturing gel as follows: 2 μl of the PCR reaction was added to 8 μl of USB stop solution (100% formamide containing xylene cyanol and bromophenol blue).

This was denatured for 5 minutes at 94° C. and transferred to an ice bucket. 3 μl was loaded on a 5% non-denaturing acrylamide gel containing 0.5×TBE and no glycerol. This was run in a 4° C. cold room in 0.5×TBE at 40 watts constant power for 2-3 hours. The gel was transferred to filter paper, dried, and autoradiographed overnight with an intensifying screen.

Results

The chromosomal localisation of the gene encoding murine Dtk has been established on chromosome 2 band F using fluorescent in situ hybridisation. This result has been confirmed using single strand conformation polymorphism analysis in the BXD recombinant inbred series.

The gene encoding human Dtk has been mapped using fluorescent in situ hybridisation to chromosome 15q15.

C. Recombinant Expression of Dtks of the Invention

In yet another aspect, the present invention relates to the recombinant expression of the Dtks or of-their extracellular receptor domains.

As will be exemplified below, the nucleic acid molecules that encode the receptors or the extracellular receptor domains of the invention may be inserted into known vectors for use in standard recombinant DNA techniques. Standard recombinant DNA techniques are those such as are described in Sambrook et al.; “Molecular Cloning” 2nd Edition Cold Spring Harbour Laboratory Press (1987) and by Ausubel et al., Eds, “Current Protocols in Molecular Biology” Greene Publishing Associates and Wiley-Interscience, New York (1987).

Vectors for expressing proteins in bacteria, especially E. coli, are known. Such vectors include the PATH vectors described by Dieckmann and Tzagoloff in J. Biol. Chem. 260, 1513-1520 (1985). These vectors contain DNA sequences that encode anthranilate synthetase (TrpE) followed by a polylinker at the carboxy terminus. Other expression vector systems are based on beta-galactosidase (pGEX); lambda P maltose binding protein (pMAL); and gluthathione S-transferase (pGST)—see Gene 67, 31(1988) and Peptide Research 3, 167 (1990).

Vectors useful in yeast are available and well known. A suitable example is the 2μ plasmid.

Suitable vectors for use in mammalian cells are also known. Such vectors include well-known derivatives of SV40, adenovirus, retrovirus-derived DNA sequences and vectors derived from combination of plasmids and phage DNA.

Further eucaryotic expression vectors are known in the art (e.g. P. J. Southern and P. Berg, J. Mol. Appl. Genet. 1, 327-341 (1982); S. Subramani et al, Mol. Cell. Biol. 1, 854-864 (1981); R. J. Kaufmann and P. A. Sharp, “Amplification And Expression of Sequences Cotransfected with a Modular Dihydrofolate Reductase Complementary DNA Gene,” J. Mol. Biol. 159, 601-621 (1982); R. J. Kaufmann and P. A. Sharp, Mol. Cell. Biol. 159, 601-664 (1982); S. I. Scahill et al, “Expression And Characterization Of The Product Of A Human Immune Interferon DNA Gene In Chinese Hamster Ovary Cells,” Proc. Natl. Acad. Sci. USA 80, 46544659 (1983); G. Urlaub and L. A. Chasin, Proc. Natl. Acad. Sci. USA 77, 4216-4220, (1980).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g. the promoter for 3-phosphoglycerate kinase, the promoters of yeast acid phosphatase, e.g. Pho5, the promoters of the yeast alpha-mating factors, and promoters derived from polyoma, adenovirus, retrovirus, and simian virus, e.g. the early and late promoters or SV40, and other sequences known to control the expression of genes of prokaryotic and eucaryotic cells and their viruses or combinations thereof.

Vectors containing the receptor-encoding DNA and control signals are inserted into a host cell for expression of the receptor. Some useful expression host cells include well-known prokaryotic and eucaryotic cells. Some suitable prokaryotic hosts include, for example, E. coli, such as E. coli SG-936, E. coli HB 101, E. coli W3110, E. coli X1776, E. coli X2282, E. coli DHT, and E. coli MR01, Pseudomonas, Bacillus, such as Bacillus subtilis, and Streptomyces. Suitable eucaryotic cells include yeast and other fungi, insect, animal cells, such as COS cells and CHO cells, human cells and plant cells in tissue culture.

A specific although non-limiting example of this aspect of the invention is set out below. It will be appreciated that while the expression of murine Dtk is exemplified, the procedures disclosed are equally applicable to the expression of other Dtks, or to the expression of extracellular receptor domains of such Dtks.

C.1 Expression of Cloned Murine Dtk in Heterologous Cell Lines

The coding region of murine Dtk was ligated in-frame into the commercially available expression vector pcDNA3 (In Vitrogen) using standard molecular biology techniques. The pcDNA3-Dtk construct was electroporated into several heterologous cell lines to demonstrate expression of Dtk. Electroporation, drug selection and isolation of Dtk-expressing clones for each cell line followed standard techniques (in M. Kriegler, “Gene Transfer and Expression—A Laboratory Manual”, Stockton Press, New York 1990).

The Dtk construct was expressed in the factor-dependent cell lines FDC-P1, BAF/3 and 32D, and in the NIH 3T3 cell line (all commercially available). The expression of Dtk in these cell lines has been ascertained at the level of RNA using standard techniques for the isolation of RNA and its detection using radiolabelled Dtk probes which are familiar to those experienced in the field (see Sambrook et al., “Molecular Cloning,” Second Edition, supra vide).

D. Lipands

The invention also includes ligands that bind to the Dtks of the invention. The ligand may be a protein such as a growth factor that occurs naturally in a mammal, preferably the same mammal that produces the corresponding Dtk. The growth factor may be isolated and purified, or be present on the surface of an isolated population of cells, such as stromal cells.

The ligand may also be a molecule that does not occur naturally in a mammal. For example, antibodies raised against the receptors of the invention or against anti-ligand antibodies mimic the shape of, and act as, ligands if they constitute the negative image of the receptor or anti-ligand antibody binding site.

Such antibodies may be polyclonal but are preferably monoclonal. Monoclonal antibodies may be produced by methods known in the art. These methods include the immunological method described by Kohler and Milstein in Nature 256, 495-497 (1975) and Campbell in “Monoclonal Antibody Technology, the Production and Characterization of Rodent and Human Hybridomas” in Burdon et al. Eds, Laboratory Techniques in Biochemistry and Molecular Biology, Volume 13, Elsevier Science Publishers, Amsterdam (1985); as well as by the recombinant DNA method described by Huse et al. in Science 246, 1275-1281 (1989).

In yet another form, the ligand may also be a non-protein molecule that acts as a ligand when it binds to, or otherwise comes into contact with, the receptor. In addition, ligands may be of two functional types. The first functional type of ligand is a molecule which binds to the receptor and stimulates it in performing its normal function (a “stimulant ligand”). The second functional type of ligand is a molecule which binds to the receptor and inhibits or prevents it performing its normal function (an “antagonistic ligand”).

Both types of ligand will find application in either therapeutic or prophylactic treatments as described below.

D.1 Sources of Ligands

The strategy for isolating a ligand for the Dtks of the invention is based on the assumption that the ligand will either be a soluble, secreted protein or alternatively it will be membrane-bound or associated.

To screen for soluble ligands, conditioned media from a range of tumor cell lines and tissues can be used. Such cell lines are readily available from the American Type Culture Collection (ATCC) Rockville, Md., USA. Conditioned media is generated from these cell lines using a variety of culture and induction protocols. The cell lines are grown using standard tissue culture techniques which are detailed by ATCC for each cell line. Conditioned medium from tissues is generated by growing minced tissue fragments in culture medium for a defined time period.

To screen for membrane-associated ligands a different approach is taken. Cell lines in which from tissues which are in close proximity to those cells or tissues which have been shown to express the Dtk receptor are used. This approach is based on the likelihood of close cell-to-cell contact between receptor-expressing cells and ligand-expressing cells. An example of this is in the testis where Sertoli cells express the receptor, while germ cells are considered a likely source of membrane-bound ligand. A further example in the brain would be where one type of neuron expresses the receptor, while microglial cells or another non-neuronal brain cell are considered likely to express the ligand.

D.2 Ligand Screening Procedures

In illustrating the screening procedures, reference will be made to murine Dtk as representative of the Dtks of the invention. Equivalent procedures can of course be employed in screening protocols using other mammalian Dtks such as human Dtk or the extracellular receptor domains of such Dtks.

Two approaches are followed to screen for the ligand for murine Dtk. If the ligand is soluble, assays which utilise either growth responses or changes in tyrosine phosphorylation will be used. Alternatively, if the ligand is membrane-bound, ligand-expressing cells will be detected using a Dtk-tag protein system whereby the extracellular domain of Dtk is fused with sequence encoding part of the human inmnunoglobulin molecule, such as the Fc region or the μ chain. The tag can then be detected using reagents which bind to the tag, such as Protein A-alkaline phosphatase or Protein A-radioiodine¹²⁵.

D.3 Soluble Ligand

To detect soluble ligand in the media conditioned by tumor cell lines or tissues, a range of concentrations of this media are added to one of the factor-dependent cell lines described above, that have been transfected with, and express the Dtk receptor. These cell lines are routinely maintained in interleukin-3 containing tissue culture medium. By withdrawing this medium and adding sources of potential ligand for Dtk, a growth response will be sought that is mediated via the introduced Dtk receptor. This response can be detected using the uptake of radiolabelled thymidine and counting this uptake by liquid scintillation spectroscopy. These techniques are standard for those familiar in the art (see Kriegler (supra); and Crosier et al., Proc. Natl. Acad. Sci. USA 88: 7744-8 (1991)).

An alternative detection system for ligands contained in tumor cell line conditioned medium uses the Dtk-expressing NIH 3T3 cell line as an indicator system, in conjunction with monitoring alterations in tyrosine phosphorylation of the Dtk receptor. Conditioned medium that contains the ligand for Dtk will trigger activation of the receptor which in turn is reflected in the phosphorylation status of the receptor. The system uses standard techniques whereby the NIH 3T3 cells are incubated with conditioned medium, cell lysates produced which in turn are immunoprecipitated with an anti-murine Dtk polyclonal antibody, proteins are resolved on SDS-PAGE gels, followed by transfer to nitrocellulose filters and subsequent Western blotting with an anti-phosphotyrosine antibody and detection using enhanced chemiluminescence techniques. These techniques are standard protein biochemistry methods (see B. Sefton and T. Hunter (eds), “Methods in Enzymology,” vol 200 and 201, 1990; and Amersham, Manufacturer's protocols for ECL techniques). The expected result with this technique would be that potential ligand-containing media would stimulate increased tyrosine phosphorylation, compared with background levels detected in these cells.

D.4 Membrane-bound Ligand

Screening for membrane-bound or associated ligands for the Dtk receptor relies on the use of a Dtk-tag fusion protein detection system. The extracellular domain of the Dtk receptor is fused in-frame to the Fc region of human immunoglobulin (IgG) or to part of the human μ chain of IgM. This procedure follows that described by Goodwin et al., Cell 73: 447456 (1993). The fusion protein is produced by transfecting the fused genes contained within the expression pEDδ c vector into COS cells. The fusion protein is purified on Protein A-Sepharose columns (Pharmacia). The Dtk-tag fusion proteins are biotinylated using sulfosuccinimiddyl-6-biotinamido)-hexanoate (Pierce Chemicals) according to the manufacturer's procedures. Alternatively, FITC-conjugated Dtk-tag fusion protein is generated by conjugating the fusion protein to FITC using standard techniques (see Suda et al., Cell 75: 169-1178, 1993).

The Dtk-tag fusion protein is used to screen for the expression of bound Dtk protein on tumor cell lines using flow cytometric techniques. The techniques used for the labelling of cells and flow cytometric analysis follow those described by Mosley et al., Cell 59: 335-348 (1989). Tumor cells are labelled on ice with the biotinylated Dtk-fusion protein using avidin-FITC, or the FTTC-labelled protein is used directly in FACS analysis. The screening procedure is aimed at detecting a cell line that produces a signal above background with the Dtk-tag fusion protein, compared with an unrelated receptor-tag fusion protein. Sequential FACS sorting of Dtk ligand-expressing cells is undertaken to generate a high Dtk ligand-expressing tumor cell subline which can be used for the generation of a cDNA expression library (for an overview of this strategy see Wong in Genetic Engineering Vol. 12, ed by J K Setlow, 1990).

E Expression Cloning of the Dtk Ligand

E.1 Construction of an Expression Library

A random-primed expression library is constructed from poly(A)⁺ mRNA isolated from the cell line or tissue demonstrated to give a positive signal in either the growth assay, phosphorylation assay or Dtk-tag fusion protein assay outlined above. The techniques used for construction of the expression library are standard procedures for those experienced in the field (see McMahon et al., EMBO J. 10, 2821-2832, 1991; and Kriegler (supra)).

E.2 Cloning of the Murine or Human Dtk Ligand

The expression library constructed from the cell line or tissue is screened by transfecting pools of cDNAs into COS cells using standard techniques (see Sambrook et al., supra). Two approaches are used to detect positive pools, depending on whether there has been evidence for either a soluble form of ligand or a membrane-bound form of ligand.

Soluble forms: COS supernatants are screened in the detection systems outlined above for soluble ligand forms. COS cells are grown in 10 cm plates using standard tissue culture techniques.

Membrane-bound forms: COS cells are grown in LabTech (Nunc) chambers and positive pools are detected by using the binding of Dtk-tag fusion protein to the COS cells, followed by detection with either a Protein A-horseradish peroxidase enzymatic reaction or Protein A-¹²⁵I binding and subsequent autoradiography.

Procedures for the breaking down of cDNA pools, subsequent sib selection and the isolation of single cDNA clones are outlined in Sambrook et al., (supra) and Wong (supra). Sequence analysis of single cDNAs follows standard techniques. Once a single cDNA clone is isolated this is transfected into COS cells or into CHO cells for large scale production of protein using standard procedures.

F Application of Ligands for the Dtks of the Invention

The types of ligand discussed above can be employed in two distinctive methods in accordance with this invention.

The first such method is a method of stimulating the proliferation, differentiation and/or survival of a cell expressing a Dtk of the invention. This stimulation, which can occur in vivo or ex vivo, involves contacting the cell with an effective amount of the ligand.

The ability of a ligand according to the invention to stimulate cells such as stem cells which express the Dtk of the invention has important therapeutic applications. Such applications include medically treating mammals, including humans, whose stem cells do not sufficiently undergo self-renewal. Examples of such medical problems which can be treated in this way include those that occur when defects in haematopoietic stem cells or their related growth factors depress the number of blood cells, leading, to disorders such as aplastic anaemia. The treatment of bone marrow damage resulting from cancer chemotherapy and radiation is another example of a medical problem that could be treated in this way.

The method can also be applied in stimulating the proliferation, differentiation and/or survival of mammalian fetal or adult neuronal cells or cells that form part of the central nervous system. Again, this has important therapeutic applications. Such applications include treating mammals, including humans, for inherited or degenerative disorders of the central nervous system. An additional application is the treatment of individuals with central nervous system trauma, for example, spinal cord trauma resulting from either crushing or asphyxia.

Yet a further therapeutic application for the ligands of the invention is in sports medicine, particularly in the treatment of muscle injuries. The Dtk of the invention is abundantly expressed on myoblast cells but not on mature muscle cells. Application of the ligand will stimulate myoblast cell proliferation and differentiation, leading to muscle repair.

In terms of ex vivo applications, the method has implications for gene therapy. In gene therapy genes are inserted into host cells (such as haematopoietic stem cells and myoblasts) and the expression of the gene regulated by either an endogenous or an exogenous promoter. However, it is often difficult to maintain growth and survival of these cells ex vivo while they are being manipulated for the insertion of foreign genes. Therefore, as the Dtk of the invention is expressed on haematopoietic stem cells and myoblasts, the ligand has a direct application in stimulating the growth, proliferation or simple survival of their cells during the manipulative process.

The second distinct method of the invention is a method of inhibiting the function of the Dtk of the invention. This method, which would normally be applied in vivo for both prophylactic and therapeutic applications, involves contacting the receptor with a ligand which blocks or prevents stimulation of the receptor (an antagonist jigand).

In terms of prophylaxis, such a method has specific application to the Sertoli cells of the testis, which abundantly express the receptor. Due to the involvement of these Sertoli cells in male fertility, contacting the receptors with an antagonistic ligand has a potential application in the control of male fertility including in male contraception.

A potential therapeutic application of contacting cells expressing the Dtk of the invention with an antagonistic ligand is in anti-tumour therapy. This potential application arises from the growing understanding of the role sometimes played by RTKs in tumour formation.

G Therapeutic Applications of Soluable Receptors

The extracellular receptor domain of the invention as described above also have potential therapeutic applications. Such applications are in a method of treating a disease, syndrome or condition caused or mediated by an excess of a ligand of the invention (whether stimulant or antagonistic).

In this method, the extracellular receptor domain of the Dtk in a soluble form can be used as a molecular “sponge” or “sink” to remove the excess of the ligand or at least to block its activity.

H Functional Equivalents

The invention includes functional equivalents of the Dtks, receptor domains, nucleic acid molecules and ligands described above.

The Dtks, extracellular receptor domains and ligands are or include proteins. A protein is considered a functional equivalent of another protein for a specific function if the equivalent protein is immunologically cross-reactive with, and has the same function as, the original protein. The equivalent may, for example, be a fragment of the protein, or a substitution, addition or deletion mutant of the protein.

For example, it is possible to substitute amino acids in a sequence with equivalent amino acids using conventional techniques. Groups of amino acids known normally to be equivalent are:

(a) Ala(A) Ser(S) Thr(T) Pro(P) Gly(G);

(b) Asn(N) Asp(D) Glu(E) Gln(Q);

(c) His(H) Arg(R) Lys(K);

(d) Met(M) Leu(L) Ile(I) Val(V); and

(e) Phe(F) Tyr(Y) Trp(W).

Substitutions, additions and/or deletions in the receptors and ligands may be made as long as the resulting equivalent receptors and ligands are immunologically cross-reactive with, and have the same function as, the native receptors and ligands.

The equivalent receptors and ligands will normally have substantially the same amino acid sequence as the native receptors and ligands. An amino acid sequence that is substantially the same as another sequence, but that differs from the other sequence by means of one or more substitutions, additions and/or deletions is considered to be an equivalent sequence. Preferably, less than 25%, more preferably less than 10%, and most preferably less than 5% of the number of amino acid residues in the amino acid sequence of the native receptors and ligands are substituted for, added to, or deleted from.

Equivalent nucleic acid molecules include nucleic acid sequences that encode equivalent receptors and ligands as defined above. Equivalent nucleic acid molecules also include nucleic acid sequences that, due to the degeneracy of the nucleic acid code, differ from native nucleic acid sequences in ways that do not affect the corresponding amino acid sequences.

Those persons skilled in the art will of course appreciate that the above description is provided by way of example only and that the invention is limited only by the lawful scope of the appended claims.

1. A mammalian receptor tyrosine kinase which is a developmental tyrosine kinase (Dtk) and which is expressed in multipotential haematopoietic cells, in embryonic stem cells, in brain tissue and in testis, but which is not expressed in mature lineage-restricted haematopoietic cells.

2. A receptor tyrosine kinase according to claim 1 that is murine Dtk having the amino acid sequence of SEQ ID NO 1, or a functional equivalent thereof.

3. A receptor tyrosine kinase according to claim 1 that is mature murine Dtk having the amino acid sequence of SEQ ID NO 2.

4. A receptor tyrosine kinase according to claim 1 that is human Dtk having the amino acid sequence of SEQ ID NO 3, or a functional equivalent thereof.

5. A receptor tyrosine kinase according to claim 1 that is mature human Dtk having the amino acid sequence of SEQ ID NO 4.

6. An extracellular receptor domain of a receptor tyrosine kinase according to claim 1.

7. An extracellular receptor domain which is the extracellular receptor domain of mature murine Dtk as defined in claim 3, or a functional equivalent thereof.

8. An extracellular receptor domain of a receptor tyrosine kinase having the amino acid sequence of SEQ ID NO 5.

9. An extracellular receptor domain which is the extracellular receptor domain of mature human Dtk as defined in claim 5, or a functional equivalent thereof.

10. An extracellular receptor domain of a receptor tyrosine kinase having the amino acid sequence of SEQ ID NO 6.

11. An extracellular receptor domain according to any one of claims 6 to 10 which is bound or attached to a support.

12. A soluble receptor comprising the extracellular receptor domain of a receptor tyrosine kinase according to any one of claims 1 to 5 lacking the transmembrane region and catalytic domain of said receptor tyrosine kinase.

13. A nucleic acid molecule encoding a receptor tyrosine kinase as defined in claim 1.

14. A nucleic acid molecule encoding murine Dtk or a functional equivalent thereof as defined in claim 2.

15. A nucleic acid molecule according to claim 14 which is DNA.

16. A DNA molecule according to claim 15 having the nucleotide sequence of SEQ ID NO 7.

17. A nucleic acid molecule encoding mature murine Dtk as defined in claim 3.

18. A nucleic acid molecule according to claim 17 which is DNA.

19. A DNA molecule according to claim 18 having the nucleotide sequence of SEQ ID NO 8.

20. A nucleic acid molecule encoding human Dtk or a functional equivalent thereof as defined in claim 4.

21. A nucleic acid molecule according to claim 20 which is DNA.

22. A DNA molecule according to claim 21 having the nucleotide sequence of SEQ ID NO 9.

23. A nucleic acid molecule encoding mature human Dtk as defined in claim 5.

24. A nucleic acid molecule according to claim 23 which is DNA.

25. A DNA molecule according to claim 24 having the nucleotide sequence of SEQ ID NO 10.

26. A nucleic acid molecule encoding an extracellular receptor domain as defined in claim 6.

27. A nucleic acid molecule encoding the extracellular receptor domain of murine Dtk or a functional equivalent thereof as defined in claim 7.

28. A nucleic acid molecule according to claim 27 which is DNA.

29. A DNA molecule according to claim 28 having the nucleotide sequence of SEQ ID NO 11.

30. A nucleic acid molecule encoding the extracellular receptor domain of human Dtk or a functional equivalent thereof as defined in claim 9.

31. A nucleic acid molecule according to claim 30 which is DNA.

32. A DNA molecule according to claim 31 having the nucleotide sequence of SEQ ID NO 12.

33. A vector including a DNA molecule a s defined in claim 13.

34. A vector including a DNA molecule as defined in any one of claims 15, 16, 18 and 19.

35. A vector including a DNA molecule as defined in any one of claims 21, 22, 24 and 25.

36. A vector including a DNA molecule as defined in claim 28 or claim 29.

37. A vector including a DNA molecule as defined in claim 31 or claim 32.

38. A method of producing a receptor tyrosine kinase comprising the steps of:
(a) culturing a host cell which has been transformed or transfected with a vector as claimed in any one of claims 33-35 to express the encoded receptor tyrosine kinase; and
(b) recovering the expressed receptor tyrosine kinase.

39. A method of producing an extracellular receptor domain of a receptor tyrosine kinase comprising the steps of:
(a) culturing a host cell which has been transformed or transfected with a vector as claimed in claim 36 or claim 37 to express the encoded extracellular receptor domain; and
(b) recovering the expressed extracellular receptor domain.

40. A recombinant receptor tyrosine kinase which is the product of a method as defined in claim 38.

41. A recombinant extracellular receptor domain which is the product of a method as defined in claim 39.

42. A ligand that binds to a receptor tyrosine kinase as defined in claim 1.

43. A ligand that binds to a receptor tyrosine kinase as defined in claim 2.

44. A ligand that binds to a receptor tyrosine kinase as defined in claim 3.

45. A ligand that binds to a receptor tyrosine kinase as defined in claim 4.

46. A ligand that binds to a receptor tyrosine kinase as defined in claim 5.

47. A ligand that binds to an extracellular receptor domain of a receptor tyrosine kinase as defined in claim 6.

48. A ligand that binds to an extracellular receptor domain of a receptor tyrosine kinase as defined in claim 7.

49. A ligand that binds to an extracellular receptor domain of a receptor tyrosine kinase as defined in claim 8.

50. A ligand that binds to an extracellular receptor domain of a receptor tyrosine kinase as defined in claim 9.

51. A ligand that binds to an extracellular receptor domain of a receptor tyrosine kinase as claimed in claim 10.

52. A ligand that binds to an extracellular receptor domain of a receptor tyrosine kinase as claimed in claim 11.

53. A ligand that binds to a soluble receptor as defined in claim 12.

54. A ligand that binds to a receptor tyrosine kinase as claimed in claim 40.

55. A ligand that binds to an extracellular receptor domain as claimed in claim 41.

56. A ligand according to any one of claims 42-55 wherein the ligand stimulates the proliferation, differentiation and/or survival of cells which express a receptor tyrosine kinase according to claim 1.

57. A ligand according to any one of claims 42-55 wherein the ligand is antagonistic and at least partially blocks or inhibits the function of a receptor tyrosine kinase according to claim 1 through binding to said receptor.

58. A method of stimulating the proliferation, differentiation and/or survival of a cell expressing a receptor tyrosine kinase according to claim 1 comprising contacting the cell with a ligand according to claim 56.

59. A method according to claim 58 wherein the stimulation occurs in vivo.

60. A method according to claim 58 wherein the stimulation occurs a vivo.

61. A method of inhibiting the function of a receptor tyrosine kinase according to claim 1 comprising contacting the receptor with a ligand according to claim 57.

62. A method according to claim 61 wherein the inhibition occurs in vivo.

63. A method according to claim 61 wherein the inhibition occurs ex vivo.

64. A method of treating a disease, syndrome or condition caused or mediated by an excess of a ligand as claimed in claim 56 comprising the step of contacting said excess of said ligand with an effective amount of a receptor tyrosine kinase according to any one of claims 1-5 and 40, an extracellular receptor domain according to any one of claims 6-11 and 41 or a soluble receptor according to claim 12.

65. A method of treating a disease, syndrome or condition caused or mediated by an excess of a ligand as defined in claim 57 comprising the step of contacting said excess of said ligand with an effective amount of a receptor tyrosine kinase according to any one of claims 1-5 and 40, an extracellular receptor domain according to any one of claims 6-11 and 41 or a soluble receptor according to claim 12.

66. A method of extracting a ligand as defined in claim 56 or claim 57 from a medium which may contain said ligand comprising the step of contacting said medium with a receptor tyrosine kinase according to any one of claims 1-5 and 40, an extracellular receptor domain according to any one of claims 6-11 and 41 or a soluble receptor according to claim 12.

67. A method of isolating ligand(s) as defined in claim 56 or claim 57 from a medium which may contain said ligand(s), comprising the steps of:
(a) contacting said medium with an effective amount of a receptor tyrosine kinase according to any one of claims 1-5 and 40, an extracellular domain according to any one of claims 6-11 and 41 or a soluble receptor according to claim 12;
(b) detecting which ligand(s) bind to said tyrosine kinase receptor, extracellular receptor domain or soluble receptor; and
(c) isolating such bound ligand(s).

68. A ligand which is isolated by a method according to claim 67.
